(19) United States
(12) Patent Application Publication
(10) Pub. No.: US 2018/0235790 A1
(43) Pub. Date: Aug. 23, 2018

(54) SYSTEMS AND METHODS FOR COUPLING AND DECOUPLING A CATHETER

(71) Applicant: BRIGHTWATER MEDICAL, INC., Dunlap, IL (US)

(72) Inventors: Harry R. SMOUSE, Dunlap, IL (US); Kent C. B. STALKER, San Marcos, CA (US); Timothy H. WILLIAMS, Palo Alto, CA (US); Edward I. WULFMAN, Woodinville, WA (US)

(21) Appl. No.: 15/901,833

(22) Filed: Feb. 21, 2018

Related U.S. Application Data
(60) Provisional application No. 62/461,443, filed on Feb. 21, 2017.

(51) Int. Cl. A61F 2/95

(52) U.S. Cl. CPC A61F 2/95; A61F 2220/0075; A61F 2250/0067; A61F 2002/9505; A61F 2220/0033; A61F 2250/0098; A61F 2210/0014

(57) ABSTRACT

A convertible catheter for use as a nephrouretheral stent, biliary stent, or other structure for maintaining patency of a bodily lumen is disclosed. After initial implantation, the proximal portion of the convertible catheter extending out from the body may simply be removed. A simple action at the catheter hub allows this proximal portion to be removed, leaving behind the implanted stent within the patient.

### CROSS-REFERENCE

This application claims priority to U.S. Provisional Application No. 62/461,443, filed Feb. 21, 2017, which is incorporated herein by reference.

The subject matter of this application is related to the subject matter of PCT Application No. PCT/US2014/063758, filed Nov. 3, 2014; and U.S. application Ser. No. 15/175,436, filed Jun. 7, 2016, which is a divisional application of U.S. patent application Ser. No. 14/159,221, filed Jan. 20, 2014 and now issued as U.S. Pat. No. 9,387,312, which is a continuation-in-part of U.S. patent application Ser. No. 12/559,946, filed Sep. 15, 2009 and now issued as U.S. Pat. No. 8,657,884, which claims priority to U.S. Provisional Patent Application No. 61/096,902, filed Sep. 15, 2008.

The subject matter of this application is also related to the subject matter of PCT Application No. PCT/US2015/044580, filed Aug. 11, 2015, which claims priority to U.S. Provisional Patent Application No. 62/036,377, filed. Aug. 12, 2014; and U.S. patent application Ser. No. 14/823,243, filed Aug. 11, 2015, which claims priority to U.S. Provisional Patent Application No. 62/036,377, filed Aug. 12, 2014.

Each of the above applications is incorporated herein by reference.

### BACKGROUND

A uretheral stent is a medical device used within a patient population which experience one or more complications associated with the urinary system which includes the kidneys, ureters, and bladder. A host of complications may affect urinary flow and how these organs handle this function; these complications ranging from decreased urine flow to swelling of the kidneys or bladder, with many of these conditions being adversely impacted by the formation of kidney stones. To alleviate urinary system complications, a device or device(s) are placed either within the bladder, one or both of the kidneys, and/or one or both of the patient's ureters. The devices used in these areas are known as nephrostomy catheters (delivered percutaneously within a kidney collecting system), nephrouretheral catheters (delivered percutaneously and extending distally into the bladder), urinary catheters (delivered through the urethra), or uretheral stents (delivered percutaneously or through the urethra).

The present disclosure relates to the delivery method and use of the nephrouretheral catheter and the uretheral stent, which are often used one after the other in percutaneous cases to deal with a patient's urinary system complications. Once a patient has exhibited urinary complications and a uretheral stent implantation is recommended, a urologically delivered stent placement will often be attempted. In some cases, this cannot be achieved by the urologist due to a variety of possible factors, resulting in the patient being sent to the interventional radiologist (IR). The IR may then attempt to deliver a nephrouretheral stent percutaneously though the backside of the patient and into the impacted kidney, with said device extending distally into the bladder. The proximal end of the nephrouretheral catheter thereby remains outside of the patient for up to 2 weeks, giving the access site sufficient time to heal before removal. Once the access site has fully healed, the patient is typically sent back to the operating room for a second interventional procedure whereby the nephrouretheral stent is removed and a uretheral stent is then delivered. This uretheral stent differs from the nephrouretheral stent in that its proximal tip terminates within the kidney's renal pelvis. This uretheral stent has a curl at its distal end which resides in the bladder and a proximal curl which resides in the renal pelvis. This device may reside in the patient for up to 6 months or in some cases longer and may be removed urologically. This two-step approach and the devices used may be less than ideal in many cases.

The present disclosure will also be related to the delivery method and use of a catheter and stent as used to maintain the patency of any bodily lumen. For example, a catheter and stent can be used to maintain the patency of a bile duct, as an ileal conduit catheter, a pancreatic stent to, e.g., drain a pancreatic pseudocyst into the stomach or intestine, to maintain the patency of a ureter where the gall bladder has been surgically removed, and at generally any part of the patient's anatomy where patency of the lumen is desired and maintained. Currently used catheters and stents to maintain bodily lumen patency may be less than ideal in at least some cases.

There are needs to overcome at least some of the drawbacks discussed above.

### SUMMARY

According to many embodiments, integrating the functionalities of two existing devices used for the percutaneous treatment of urinary and other complications (e.g., maintaining the patency of a bile duct, or draining various cysts) into a single device has been devised, with particular focus on the methods, designs, and materials which may be utilized to couple these two devices together in a fashion which allows a decoupling at a later time state. Many embodiments provide a single device which may combine the functionalities of a nephrouretheral or other catheter and uretheral or other stent, but can maintain the ability to perform the full removal of the proximal (catheter) portion of the device extending out of the patient's body during the early stages of implantation (for example, up to 2 weeks).

The decoupling (release) mechanism can allow the proximal portion of this combination device to be removed without the need for a second interventional procedure. The primary modes of function of this coupling mechanism include, but are not limited to, the following: (1) to maintain connection of proximal (catheter) portion of device to distal (stent) portion of device, and (2) to permit the removal of the proximal portion of device at a later time leaving behind distal portion of device within the patient's urinary collecting system or the patient's desired anatomical location. The decoupling nature of the proximal portion of the device may be achieved by providing an input to the proximal hub of the device which extends out of the patient's body. This input to decouple catheter from stent may be performed by the push of a button, the rotation of a luer, the insertion of a tool, the removal of a wire or a series of similar events occurring at the proximal hub, or the like. Additionally, independently of the coupling mechanism, a strand of material, typically with a circular cross-section, can be used to assist in the closure of the stent's proximal loop once the device has been delivered into the patient. This may be necessary due to the tighter space the renal pelvis or other target organ provides for this proximal loop to reside. This strand of material may be called the ‘proximal loop suture’ and may pass through side holes cut into the stent allowing for proximal loop closure. This ‘proximal loop suture’ may be fully removed from the device without inhibiting the functionality of the coupling interface between the proximal and distal portions of the device.

Several depictions of the coupling interface between the catheter and stent are shown in the Figures. This coupling interface would permit the utilization of a single surgical procedure as opposed to two, putting the patient at significantly less risk for complications in the operating room environment. The decoupling may be achieved by an input to the proximal hub performed at bedside or by the insertion of a decoupling tool, thereby removing catheter portion of device once deemed necessary. A coupled device may be achieved in many ways as described herein. An example of a coupling may include an expandable inner member which retains the distal member with the proximal member by expanding within the stent lumen to couple and once an input is applied to proximal hub, said expanded element may collapse and decouple the device. Some of the depictions below may provide a safe and effective way to combine the nephrouretheral or other catheter and the uretheral or other stent while still providing the utility of separate devices and two surgical procedures.

Aspects of the present disclosure provide surgically delivered medical devices. An exemplary medical device may comprise a proximal portion which extends outside of a patient's surgical access site. The proximal portion of device may be removed at a later date, converting the distal portion of device into an implant. The device may comprise a distal (stent) member and a proximal (catheter) member. The proximal and distal members may be coupled to one another in a concentric fashion via an inner member extending out from the proximal member. The proximal and distal members may be coupled in one of or a combination of many embodiments.

In many embodiments, the device may employ suture loop lock(s) to couple the proximal member to the distal member. The suture loop lock(s) may wrap around one or more pull wire(s) at the inner member to stent interface. Furthermore, suture tail(s) may extend proximally to the hub of device and may be locked into place with tension applied to achieve leveraged coupled interface.

In many embodiments, the device may employ suture loop lock(s) which wrap around the inner member at the stent interface region to achieve coupling of proximal and distal members. Furthermore, suture tail(s) may extend proximally to hub of device and may lock into place with tension applied to achieve leveraged coupled interface.

In many embodiments, the device may comprise an inner member which is fixed at the distal region of proximal catheter. The inner member may contain a smaller tube affixed within its lumen. The smaller tube may be used as a receiver for a ball wire, which may extend from distal member, and a pull wire, which may extend from proximal member. Once the ball wire has passed through the smaller tube, the pull wire may be passed through which may prevent passing of ball until pull wire is removed from device.

In many embodiments, the device may comprise an inner member which is fixed at the distal region of the proximal catheter. The inner member may include a superelastic/shape memory element which may be used as a receiver described above.

In many embodiments, the proximal and distal members of the device may be coupled to one another using a ring locking style mechanism, with one ring element affixed to distal member and another ring element affixed to proximal member. The ring members may be held coincident using an inner member and a pull wire.

In many embodiments, the device may comprise a keyed locking system, such as mating hexagonal elements, with one hex element affixed to proximal member and another hex element affixed to distal member to achieve coupling. The hex elements may be engaged or disengaged using a counter rotating tool.

In many embodiments, the inner member may extend fully from proximal hub to achieve concentric junction between the distal and proximal members. In addition, the inner member may be fixed or movable at hub and along entire catheter length.

In many embodiments, the inner member may be a component which is affixed to the distal or proximal member and only extends for a fractional portion of the device's length.

In many embodiments, the inner member may be formed as a necking of the distal region of the catheter itself which is then inserted into the lumen of the distal (stent) member.

In many embodiments, the proximal and distal members of the device may be coupled to one another through the employment of an adhesive layer on the inner member region which extends into the distal member.

In many embodiments, the proximal and distal members of the device may be coupled to one another through the employment of an oversized diameter of the inner member resulting in a frictional fit with the stent.

In many embodiments, the proximal and distal members of the device may be coupled to one another through the employment of a metallic or polymeric crimp which may be applied to the outside of the stent which overlaps the inner member extending into its lumen.

In many embodiments, the proximal and distal members of the device may be coupled to one another through the employment of a superelastic/shape memory alloy affixed to the distal member which may interface with protrusions on the outer surface of the inner member. Thereby, the inner member may not be movable until the catheter or peel-away sheath has been removed and shape memory alloy mechanism releases inner member.

In many embodiments, an inner tube extends inside and along the lumen of the catheter and the lumen of the stent. The proximal and distal members of the device may be coupled to each other by means of a flap on the catheter fitted into a complementary shaped slot on the stent. The flap may be biased radially inward toward the lumen, and thereby be abutting the slot when the catheter and stent are pulled apart while the inner tube is present in the lumens thereof (i.e., crosses through the lumens of both the catheter and the stent), preventing the proximal and distal members from decoupling. When the inner member is retracted, the flap may be allowed to return to its radially inward oriented position such that it no longer abuts the slot such that the catheter and stent may be decoupled. In other embodiments, the flap may instead be on the stent and the complementary shaped slot may be on the catheter.

In many embodiments, the proximal and distal members of the device may be coupled to one another through the use of a mechanically modified surface of the inner member which, once inserted into distal member, an interfacing region of the distal member may be heated and a polymer may be allowed to flow into the mechanical alterations of inner member. The polymer may furthermore be allowed to cool, forming a permanent mechanical interface between the two elements until the inner member is pulled away from distal member using a light to moderate pull force.

In many embodiments, the proximal and distal members of the device may be coupled to one another through the use of a female to male thread style arrangement at the coupling interface.

In many embodiments, the proximal and distal members of the device may be coupled to one another using electrically releasable metallic element(s), which may couple the proximal and distal members until a tool can be used to electrically disengage said elements.

In many embodiments, the proximal and distal members of the device may be coupled to one another using magnets affixed to proximal and distal members and may be disengaged by pulling proximal member away from distal member or by rotating one or both of magnetic components within said members using a tool or other components incorporated within device.

In many embodiments, the proximal and distal members of the device in their coupled state may be disengaged using a separate tool which may decouple proximal and distal members by an input of rotation, electrical stimulus or ultrasonic vibration.

Aspects of the present disclosure also provide further stent delivery systems. An exemplary stent delivery system may comprise a catheter body, a stent member, an inner member, and a tether. The catheter body may have an inner lumen and a proximal end and a distal end. The stent member may have an inner lumen and a proximal end releasably coupled with the distal end of the catheter body. The inner member assembly may be disposed in the inner lumen of the catheter body and may extend into the inner lumen of the stent member to concentrically align the catheter body and the stent member. The tether may extend through or along the catheter body and into the inner lumen of the stent member to form a loop over at least a portion of the inner member assembly, thereby securing the stent member to the catheter body. Retraction of the inner member from the inner lumen of the stent member may free the inner member assembly from the loop such that the stent member is released from the stent body.

The inner member assembly may comprise a locking pull wire. The locking pull wire may be threadable through the loop of the tether. The inner member assembly may comprise a hypotube. The inner member assembly may be configured to be actuated with one or more pull tabs or rotatable caps at a hub coupled to the proximal end of the catheter body.

The tether may extend through the inner lumen of the catheter body. The tether may extend out of a lateral port of the catheter body near the distal end of the catheter body. The loop formed by the tether may extend into stent member through a lateral port of the stent member to be threaded through by the at least a portion of the inner member assembly within the inner lumen of the stent member. The tether may have a fixed end near the distal end of the catheter body and a free end. The tether may extend proximally toward the free end and the proximal end of the catheter body. The tether may have a first end and a second end. The tether may extend proximally toward both the first and second ends and the proximal end of the catheter body.

The stent member may comprise a proximal loop and a distal loop. One or more of the proximal loop or the distal loop of the stent member may have a straightened configuration and a looped configuration. One or more of the proximal loop or the distal loop may be biased to assume the looped configuration. The stent delivery system may further comprise a loop pull wire extending through the inner lumen of the catheter body and coupled to the proximal loop. Retracting the loop pull wire may pull the proximal loop into the loop configuration or may lower a radius of the proximal loop. The loop pull wire may extend out from a first lateral port of the stent member near the proximal end of the stent member and may extend back into a second lateral port of the stent member near a distal end of the proximal loop. The loop pull wire may be retractable from a pull tab or rotatable cap at a hub coupled to the proximal end of the catheter body.

Other exemplary stent delivery systems may comprise a catheter body, a catheter member, and an inner member assembly. The catheter body may have an inner lumen and a proximal end and a distal end. The catheter member may have an inner lumen and a proximal end which is fixed or releasably coupled with a stent element extending from within the lumen of the proximal end of the stent body. The inner member assembly may be disposed in the inner lumen of the catheter body and may extend into the inner lumen of the stent member to concentrically align the catheter body and the stent member.

In some embodiments, the stent delivery system further comprises a wire extending through or along the entire or a portion of the catheter body and into the inner lumen of the stent body to interface the catheter member, with the stent element thereby securing the stent body to the catheter body. Retraction of the wire from the inner lumen of the catheter member may free the inner member assembly from the stent element such that the catheter member is released from the stent body.

In some embodiments, the stent delivery system may further comprise a wire extending through or along the entire or a portion of the catheter body and into the inner lumen of the stent member, subsequently interfacing with the superelastic assembly in a releasable fashion to secure the stent member to the catheter body. Retraction of the wire from the inner lumen of the stent member may free the superelastic inner member assembly from such that the stent member is released from the stent body.

In some embodiments, the stent delivery system may further comprise a tether extending through or along the catheter body and into the inner lumen of the stent member to form a loop over at least a portion of the inner member assembly, thereby securing the stent member to the catheter body. Retraction of the inner member from the inner lumen of the stent member may free the inner member assembly from the loop such that the stent member is released from the stent body.

In some embodiments, the stent delivery system may further comprise a tether extending through or along the catheter body and into the inner lumen of the stent member to form a loop over at least a portion of the inner member assembly, thereby securing the stent member to the catheter body. Retraction of the inner member from the inner lumen of the stent member may free the inner member assembly from the loop such that the stent member is released from the stent body.

In some embodiments, the stent delivery system may further comprise an adhesive which is applied to the inner lumen of the stent member to affix the inner member assembly to the stent member extending through or along the catheter body and into the inner lumen of the stent member, thereby securing the stent member to the catheter body. Retraction of the inner member at a threshold load may allow a break away from the bonded surface of the stent member such that the inner member is released from the stent body.

In some embodiments, the stent delivery system may further comprise a frictional interference between the inner member and the stent member. The frictional interference may be applied to the inner lumen of the stent member to affix the inner member assembly to the stent member extending through or along the catheter body and into the inner lumen of the stent member, thereby securing the stent member to the catheter body. Retraction of the inner member at a threshold load may allow a breakaway of the frictional interference with the stent member such that the inner member is released from the stent body.

In some embodiments, the stent delivery system may further comprise a metallic crimp or swaged band element. The metallic crimp or swaged band element may be applied over the outside of the stent body toward its distal end to affix the inner member assembly to the stent member extending through or along the catheter body and into the inner lumen of the stent member thereby securing the stent member to the catheter body. Retraction of the inner member at a threshold load may allow a breakaway from the frictional interference resulting from the crimp element such that the inner member is released from the stent body.

In some embodiments, the stent delivery system may further comprise a superelastic mechanism extending from the stent body. The superelastic mechanism may interface with the inner member in a locked state until the catheter body is removed, at which point the superelastic mechanism may release the inner member from its locked state allowing its complete removal.

In some embodiments, the stent delivery system may further comprise a thermoforming process applied to the inner member allowing it to interface with the stent member to affix the inner member assembly to the stent member extending through or along the catheter body and into the inner lumen of the stent member, thereby securing the stent member to the catheter body. Retraction of the inner member at a threshold load may allow a breakaway from the thermoformed surface of the stent member such that the inner member is released from the stent body.

In some embodiments, an inner member and stent member may interface and lock together via threaded surfaces to affix the inner member assembly to the stent member extending through or along the catheter body and into the inner lumen of the stent member, thereby securing the stent member to the catheter body. Rotation of the inner member out from the stent member may enable inner member to be released from the stent body.

The stent delivery systems may further be configured in any number of ways described above and herein.

Aspects of the present disclosure also provide methods for delivering nephrouretheral or other stents. A stent delivery system may be advanced through a percutaneous access site so that a distal end of a stent member of the stent delivery system is positioned in a bladder or other target organ and a proximal end of the stent member is positioned in a renal pelvis or other target organ. The distal end of the stent member may form a distal loop in the bladder or other target organ. The proximal end of stent member may be actuated to form a proximal loop in the renal pelvis or other target organ. The stent member may be decoupled from a catheter body of the stent delivery system. The catheter body of the stent delivery system may be retracted from the percutaneous access site, leaving the stent member in place.

To actuate the proximal end of the stent member to form a proximal loop in the renal pelvis or other target organ, a loop pull wire extending through the catheter body may be retracted to reduce a radius of the proximal end of the stent member.

To decouple the stent member from the catheter body, a lock pull wire may be retracted from the stent member to free a tether loop extending into the stent member from the catheter body and/or an inner member may be retracted from the stent member. The inner member may be configured to concentrically align the catheter body with the stent member when advanced therethrough.

The member and the catheter body of the stent delivery system may be left in place for at least 3 days before the stent member is decoupled from the catheter body and the catheter body is retracted from the percutaneous access site. In some embodiments, urine is be drained through the catheter body of the stent left in place. In some embodiments, the catheter body of the stent left in place is capped.

Aspects of the present disclosure also provide methods of delivering a convertible stent. A stent delivery system may be advanced through a percutaneous access site so that a distal end of a stent member of the stent delivery system is positioned in an intestine, and a proximal end of the stent member is positioned in a common bile duct, wherein the distal end of the stent member forms a distal loop in the intestine. The proximal end of stent member may be actuated to form the proximal end into a proximal loop in the common bile duct. The stent member may be decoupled from a catheter body of the stent delivery system. The catheter body may be retracted from the percutaneous access site, leaving the stent member in place.

In some embodiments, a loop suture may be pulled to actuate the proximal end of the stent member to form the proximal loop.

In some embodiments, one or more of the proximal or distal end of the stent member may be straightened prior to advancing the stent delivery system.

In some embodiments, to straighten the one or more of the proximal or distal end of the stent member, a hypotube or other straightener may be advanced through inner lumens of the stent member and the catheter body

In some embodiments, the hypotube or other straightener may be removed, thereby allowing the one or more of the proximal or distal end of the stent member to form the proximal loop or the distal loop, respectively.

In some embodiments, the stent member and the catheter body of the stent delivery system may be left in place until hemostasis is achieved, before decoupling the stent member from the catheter body.

In some embodiments, at least a portion of the catheter body may be coated with a hemostatic agent.

Aspects of the present disclosure also provide further methods of delivering a convertible stent. A stent delivery system may be advanced through a percutaneous access site so that a distal end of a stent member of the stent delivery system is positioned in a pseudocyst, and a proximal end of the stent member is positioned in a stomach, wherein the distal end of the stent member forms a distal loop in the pseudocyst. The proximal end of stent member may be actuated to form the proximal end of the stent member into a proximal loop in the stomach. The stent member may be decoupled from a catheter body of the stent delivery system. The catheter body may be retracted from the percutaneous access site, leaving the stent member in place.

In some embodiments, a loop suture may be pulled to actuate the proximal end of the stent member to form the proximal loop.

In some embodiments, one or more of the proximal or distal end of the stent member may be straightened prior to advancing the stent delivery system.

In some embodiments, to straighten the one or more of the proximal or distal end of the stent member, a hypotube, or other straightener may be advanced through inner lumens of the stent member and the catheter body.

In some embodiments, the hypotube or other straightener may be removed, thereby allowing the one or more of the proximal or distal end of the stent member to form the proximal loop or the distal loop, respectively.

In some embodiments, the stent member and the catheter body of the stent delivery system may be left in place until hemostasis is achieved, before decoupling the stent member from the catheter body.

In some embodiments, at least a portion of the catheter body may be coated with a hemostatic agent.

Aspects of the present disclosure also provide further stent delivery systems. An exemplary stent delivery system may comprise a catheter body, a stent member, and an inner member. The catheter body may have a lumen and a terminal end and a flap at the terminal end, the flap being biased radially inward toward the lumen. The stent member may have a lumen and a terminal end and a slot at the terminal end, the slot having a shape complementary to the flap of the catheter body to receive the flap. The inner member may be translatable through the lumens of the catheter body and the stent member. The catheter body and the stent member may be coupled to one another by matching the terminal ends thereof to one another and crossing the inner member between the lumens of the catheter body and the stent member, with the flap being received by the slot to restrict separation of the catheter body and the stent member, and the inner member pushing the flap radially outward to maintain a position of the flap within the slot.

Aspects of the present disclosure also provide further stent delivery systems. An exemplary stent delivery system may comprise a catheter body, a stent member, and an inner member. The catheter body may have an inner lumen and may be configured to couple to the stent member. The stent member may have an inner lumen and may be configured to couple to the catheter body. The stent member may comprise a proximal loop and a distal loop. The inner member assembly may extend through the inner lumens of the catheter body and the stent member, concentrically aligning and coupling the catheter body and the stent member to one another.

In some embodiments, the distance between the proximal loop and the distal loop may be between 25 cm and 35 cm. In some embodiments, the distance between the proximal loop and the distal loop may be between 20 cm to 28 cm. The distance between the proximal loop and the distal loop may generally be any length appropriate for a desired application. The distance ranges of 25 cm to 35 cm and 20 cm to 28 cm may be appropriate for use of the convertible stent as a nephrouretheral stent.

In some embodiments, the distance between the proximal loop and the distal loop may be between 5 cm and 10 cm. which may be appropriate for use of the convertible stent as a biliary stent.

In some embodiments, the catheter body and stent member may be tapered distally, i.e., gradually tapered to facilitate advancement.

In some embodiments, a diameter of the catheter body may be larger than a diameter of the stent member at the proximal loop, and a diameter of the stent member at the proximal loop may be larger than a diameter of the stent member at the distal loop.

In some embodiments, the diameter of the catheter body may be 10F, the diameter of the stent member at the proximal loop may be 9F, and the diameter of the stent member at the distal loop may be 8F. The different portions of the assembly of the catheter and stent members may instead have other dimensions or diameters to provide the gradual tapering in the distal direction.

In some embodiments, the stent member may comprise polymer, UHMW polyethylene, PTFE, or any combination thereof.

In some embodiments, the inner member assembly comprises a stent inner tube and a catheter inner tube. The stent inner tube may be coupled to the stent member and disposed within the inner lumen of the stent member near a proximal end of the stent member. The stent inner tube may comprise a lock window. The catheter inner tube may be coupled to and protruding from a distal end of the catheter member. The catheter inner tube may comprise a locking element. The catheter inner tube may be configured to fit at least partially within the stent inner tube such that the locking element is in a position to be urged to fit within the lock window of the stent inner tube, thereby coupling the catheter body with the stent member.

In some embodiments, the catheter inner tube comprises a strut coupled to the locking element, the strut being biased to urge the locking element radially inward to be away from the lock window when the catheter inner tube is fit at least partially within the stent inner tube. The inner member assembly may comprises a slider tube configured to be advanced through an inner lumen of the catheter inner tube to urge the lock element radially outward to fit within the lock window of the stent inner tube when the catheter inner tube is fit at least partially within the stent inner tube. The slider tube may further comprise a pull wire configured to be retracted to retract the slider tube from the inner lumen of the catheter inner tube.

In some embodiments, the inner member assembly further comprises a stent outer tube coupled to the stent member and positioned within the inner lumen of the stent member to couple the stent inner tube to the stent member.

In some embodiments, the inner member assembly further comprises a catheter outer tube coupled to the catheter member and positioned within the inner lumen of the catheter member to couple the catheter inner tube to the catheter member.

Aspects of the present disclosure also provide further methods of delivering a convertible stent. A stent delivery system may be advanced through a percutaneous access site so that a distal end of a stent member of the stent delivery system is positioned in first bodily lumen, a proximal end of the stent member is positioned in a second bodily lumen, and a catheter member proximal to the stent member and coupled thereto is positioned in a tissue tract leading to the first and second bodily lumens. The distal end of the stent member may be allowed to form a distal loop. The proximal end of the stent member may be allowed to form a proximal loop. Bleeding in the tissue tract may be reduced using a hemostatic element of the catheter member. The stent member may be decoupled from the catheter member. The catheter member may be retracted from the percutaneous access site, leaving the stent member in place.

In some embodiments, the hemostatic element may comprise a hemostatic coating on the catheter member.

In some embodiments, the hemostatic element may comprise an electrocauterizing electrode.

In some embodiments, a loop suture may be pulled to actuate the proximal end of the stent member to form the proximal loop.

In some embodiments, one or more of the proximal or distal end of the stent member may be straightened prior to advancing the stent delivery system.

In some embodiments, to straighten the one or more of the proximal or distal end of the stent member, a hypotube, or other straightener may be advanced through inner lumens of the stent member and the catheter body.

In some embodiments, the hypotube or other straightener may be removed, thereby allowing the one or more of the proximal or distal end of the stent member to form the proximal loop or the distal loop, respectively.

In some embodiments, the stent member and the catheter body of the stent delivery system may be left in place until hemostasis is achieved, before decoupling the stent member from the catheter body.

While the present disclosure describes the device in the context of a ureter obstruction, this is for example only. The device of may also be used in other contexts where it is desired to maintain the patency of a lumen. For example, the device may be used as a device which converts into a biliary stent to maintain the patency of a bile duct, a pancreatic stent to, for example, drain a pancreatic pseudocyst into the stomach or intestine, a nephrouretal stent where the bladder has been surgically removed, or generally with any part of the body where patency of a lumen is desirable and is to be maintained.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings which display various embodiments of the coupling mechanism to be used in the fabrication of the convertible nephrouretheral catheter and are described as follows.

FIG. 1 is a side view of an example convertible nephrouretheral catheter, according to many embodiments.

FIG. 2 is a side section view detailing two coupling methods utilizing the principle of a suture loop lock in conjunction with a pull wire, according to many embodiments.

FIGS. 3A and 3B are side section views of a suture loop lock similar to that of the previous figures, but where the inner member of the catheter is grabbed by the suture loop(s) as opposed to using a pull wire, according to many embodiments.

FIGS. 4A, 4B, 4C, and 4D are side section views of the coupling region between the catheter (proximal member) and stent (distal member) with elements that have been affixed to both proximal and distal members which may be used for coupling and decoupling of said members (with decoupling means achieved by removing pull wire from assembled elements), according to many embodiments.

FIGS. 5A and 5B are perspective and side section views, respectively, of the coupling region which may utilize a superelastic/shape memory alloy affixed to proximal and/or distal member, according to many embodiments.

FIGS. 6A, 6B, and 6C are perspective views of a coupling region which utilizes locking ring elements to join proximal and distal members; and, FIG. 6D is a side section view of this coupling region.

FIG. 7A is a side view of a coupling region which utilizes hexagonal elements affixed to proximal and distal members to achieve coupling; FIG. 7B shows a front section view of the coupling region; FIG. 7C shows a side view of the coupling region; FIG. 7D shows a perspective view of the coupling region; FIG. 7E shows a tool that may be used to actuate the hexagonal elements in the coupling region; and FIG. 7F shows a handle mechanism for the tool, according to many embodiments.

FIGS. 8A, 8B, and 8C shows side views of coupling configurations, according to many embodiments.

FIG. 9 shows a side section view of the application of an adhesive on inner member of catheter to achieve coupling between proximal and distal members, according to many embodiments.

FIG. 10 shows a side section view of a friction/press fitting of the inner member of the catheter into the lumen of the stent to achieve coupling of proximal and distal members, according to many embodiments.

FIGS. 11A and 11B show side views of a metallic element applied to the outer surface of the stent to crimp a catheter inner member to achieve coupled state, according to many embodiments.

FIGS. 12A and 12B show side views of the use of a superelastic/shape memory alloy affixed to stent used to couple proximal and distal members, according to many embodiments.

FIG. 12C shows a side view of a flap and a complimentary slot used to couple the proximal and distal members, along with an inner member, according to many embodiments.

FIGS. 12D-12F shows top views of exemplary shapes of a flap and exemplary shapes of a slot used to couple the proximal and distal members, according to many embodiments.

FIG. 12G shows a side view of the inner member removed from the proximal and distal portions, such that the proximal and distal portions are decoupled, with the flap biased inward toward the lumen, according to many embodiments.

FIGS. 13A and 13B show side views of a method to couple proximal and distal members by thermally processing a region of stent, according to many embodiments.

FIG. 14 shows a side section view detailing a threaded coupling whereby distal member (stent) has female threads (inner surface) and proximal member (catheter) has male threads over its outer surface within the coupling region, according to many embodiments.

FIGS. 15A, 15B, and 15C show side views displaying the proximal loop suture and its independent functionality from the coupling mechanism, according to many embodiments.

FIGS. 16A, 16B, and 16C show side views detailing several proximal hub configurations, according to many embodiments.

FIG. 17A shows a side view of a nephrouretheral stent system, according to many embodiments.

FIG. 17B show a side view of the nephrouretheral stent system of FIG. 17A with the stent member detached.

FIG. 17C shows a side section view of the coupling and release mechanism of the nephrouretheral stent system of FIG. 17A.

FIG. 17D shows a perspective view of the lock/release wire of the coupling and release mechanism of FIG. 17C.

FIG. 17E shows a side view of the nephrouretheral stent system of FIG. 17A.

FIG. 17F shows a magnified view of the proximal loop of the stent member of the nephrouretheral stent system of FIG. 17A.

FIGS. 18A, 18B, and 18C show side section views of a coupling and release mechanism releasing the stent member of a nephrouretheral stent system, according to many embodiments.

FIG. 19 shows a side section view of a side section view of a coupling and release mechanism for a nephrouretheral stent system, according to many embodiments.

FIG. 20 shows a side section view of a side section view of a coupling and release mechanism for a nephrouretheral stent system, according to many embodiments.

FIG. 21 shows a side section view of a side section view of a coupling and release mechanism for a nephrouretheral stent system, according to many embodiments.

FIGS. 22A and 22B show perspective and perspective side section views of an exemplary hub for a nephrouretheral stent system, according to many embodiments.

FIGS. 23A-23D show another exemplary hub for a nephrouretheral stent system, according to many embodiments. FIGS. 23A, 23B, and 23C show perspective views of the hub. FIG. 23A shows the hub being axially collapsed, FIG. 23B shows the hub being partially separated, and FIG. 23C shows the hub being fully separated out so that multiple pull tabs can be used. FIG. 23D shows a side section view of the hub.

FIG. 24 shows further exemplary hubs for nephrouretheral stent systems, according to many embodiments.

FIG. 25A show perspective views of various rotating hemostat type valves usable for various convertible stent systems, according to many embodiments.

FIG. 25B shows a perspective view of a center lever lock usable for various convertible stent systems, according to many embodiments.

FIG. 26 shows a section view of a convertible stent system showing the arrangement of its loop suture, according to many embodiments.

FIGS. 27A-27F show a method of inserting and using a convertible stent system into the kidney of a patient, according to many embodiments. FIG. 27A shows a schematic of the relevant anatomy; FIG. 27B shows the convertible stent percutaneously inserted into the kidney and bladder as straightened; FIG. 27C shows the loops of the convertible stent being reintroduced; FIG. 27D shows the convertible stent implanted in the kidney and bladder to drain urine externally; and FIGS. 27E and 27F show the catheter portion of the convertible stent being removed, leaving the stent portion to maintain patency of the ureter between the renal pelvis and the bladder.

FIGS. 28A-28D show a method of inserting and using a convertible stent system into the common bile duct of a patient, according to many embodiments. FIG. 28A shows a schematic of the relevant anatomy; FIG. 28B shows the convertible stent percutaneously inserted through the liver and into the common bile duct and small intestine as straightened; FIG. 28C shows the loops of the convertible stent being introduced and the catheter portion of the convertible stent being removed; and FIG. 28D shows the stent portion as implanted to maintain patency of the common bile duct.

FIGS. 29A-29D show a method of inserting and using a convertible stent system into the pseudocyst of a patient, according to many embodiments. FIG. 29A shows a schematic of the relevant anatomy; FIG. 28B shows the convertible stent percutaneously inserted through the liver and into the common bile duct and small intestine as straightened; FIG. 28C shows the loops of the convertible stent being introduced and the catheter portion of the convertible stent being removed; and FIG. 28D shows the stent portion as implanted to maintain patency of the common bile duct.

FIGS. 30A-30F show additional embodiments of a coupling and release mechanism, according to many embodiments. FIG. 30A shows a perspective view of the catheter side of the coupling and release mechanism. FIGS. 30B1, 30B2, and 30B3 show perspective views of an outer tube, inner tube assembly, sliding tube assembly, respectively of the catheter side of the coupling and release mechanism. FIGS. 30B4 and 30B5 show perspective views of the outer tube and inner tube, respectively, of the stent side of the coupling and release mechanism. FIG. 30C shows a top view of the inner tube of the catheter side of the coupling and release mechanism, including cutting and welding paths to manufacture the inner tube. FIG. 30D shows a cut-away, perspective view of the outer tube, inner tube assembly, and the sliding tube assembly of the catheter side of the coupling and release mechanism being coupled together. FIG. 30E shows a cut-away, perspective view of the outer tube and inner tube of the stent side of the coupling and release mechanism being coupled together. FIG. 30F shows a cross-sectional side view of the coupling and release mechanism coupling the catheter and stent sides of the convertible stent together.

FIGS. 31A-31F show side sectional views displaying various embodiments of the proximal loop suture and their loop forming and straightening mechanisms, according to many embodiments

### DETAILED DESCRIPTION

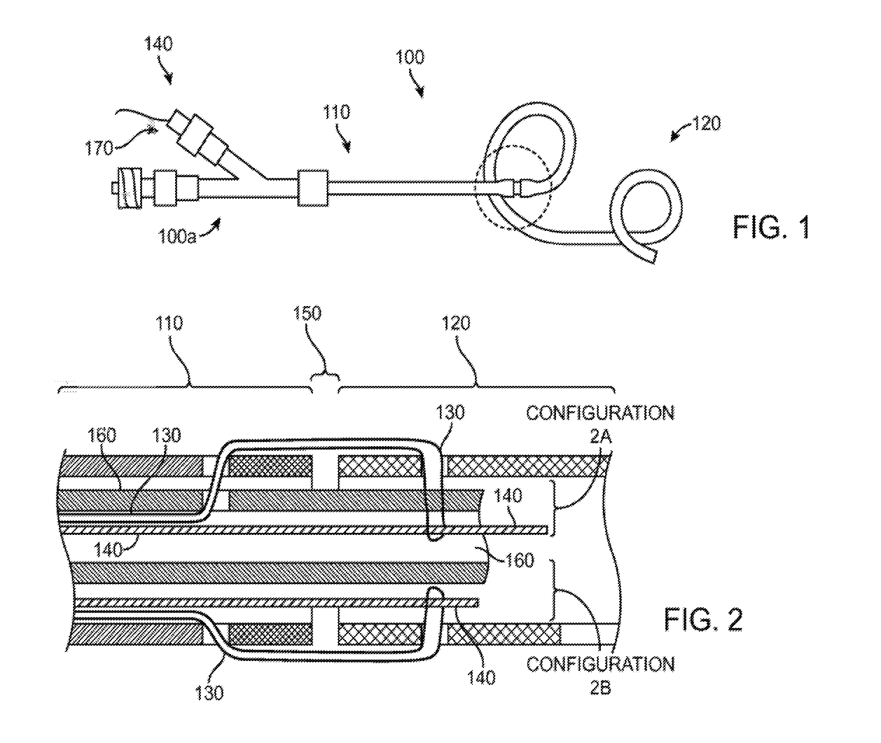

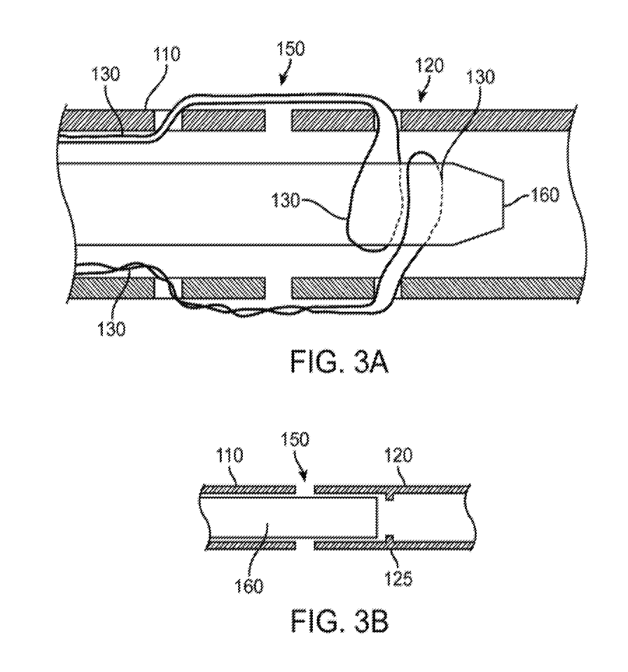

FIG. 1 provides a perspective of an example configuration of the convertible nephrouretheral catheter 100. FIG. 2 details the coupling region of proximal member (catheter) 110 and distal member (stent) 120 with two example configurations shown. The release mechanism shown may comprise two elongated members—suture 130 and pull wire 140. As shown in FIG. 2, the suture 130 may exit through the wall of the proximal member 110 and reenter the distal member 120, holding the proximal and distal members 110, 120 together across the junction 150. The pull wire 140 can keep the suture 130 from pulling out until the pull wire (lock wire) 140 is removed or proximally retracted. The distal portion of the suture 130 may form a loop through which the distal portion of the pull wire 140 is threaded through. Additionally, an inner-member 160 may be included to cross the junction 150 on the inside to keep junction aligned (concentric) and facilitate passage of other components across the junction 150. Various configurations are shown in FIG. 2, where the suture 130 can be inside the inner member 160 and pass through the inner member 160 in addition to proximal and distal members 110, 120 (Configuration 2A). Alternatively or in combination, the suture 130 can be outside the inner member 160 and pass through only the proximal and distal members 110, 120 (Configuration 2B). Both the pull wire 140 and the suture 130 may be made of various suitable materials and shapes of materials, as well as other components. To release the locking suture 130, the pull wire 140 may be pulled proximally from a pull tab 170 on the handle portion or proximal hub 100a of the convertible catheter 100. FIGS. 3A and 3B detail an additional configuration of the suture loop 130 lock(s) which may wrap around inner member 160 as opposed to utilizing a pull wire 140 to achieve coupling. The distal portion of the suture 130 may form a loop through which the distal portion of the inner member 160 is threaded through (FIG. 3A). When the inner member 160 is retracted proximally, the distal loops of the suture 130 may be released which allows the proximal and distal members 110, 120 to separate. In some embodiments, the inner member 160, the proximal member 110, and the distal member 120 may form an interference fit with one another at the junction 150 to prevent displacement of the proximal and distal members 110, 120, although this interference fit may be decoupled by retraction of the inner member 160 relative to the junction 150. In some embodiments, the distal member 120 may comprise stops 125 along the inner surface of its lumen to restrict the distal advancement of the inner member 160 into the lumen (FIG. 3B).

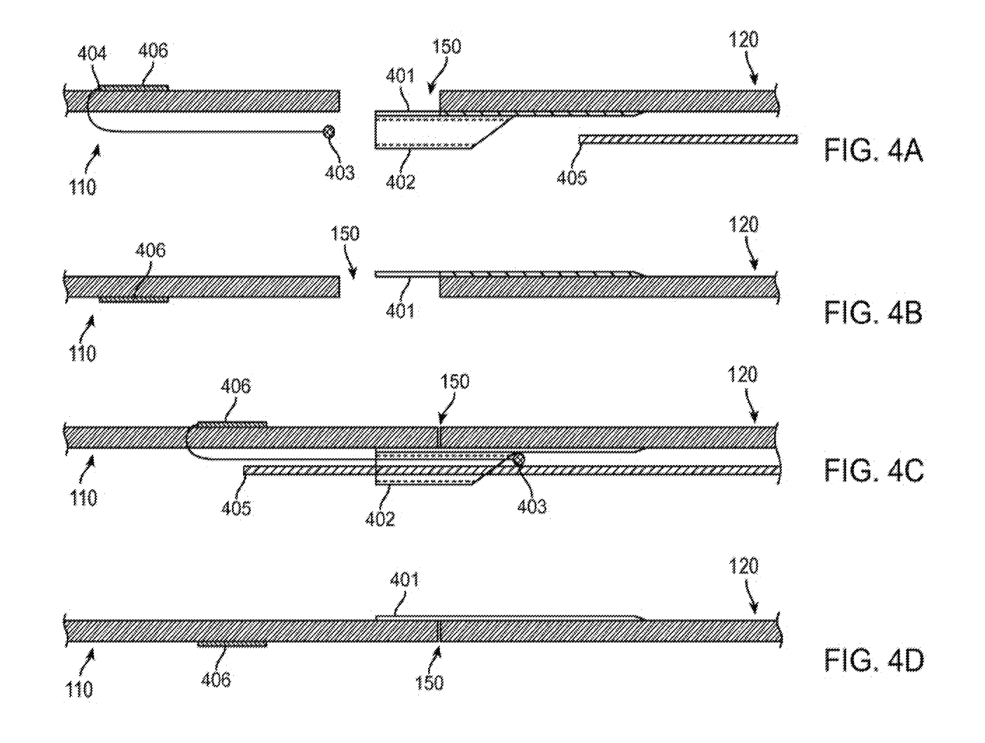

FIGS. 4A-4D display enhanced perspectives of the coupling region between distal (stent) members 120 and proximal (catheter) members 110 of the device 100. Various elements which may be fabricated from metallic or polymeric materials, may be affixed to the coupling region of the proximal member 110. Shown by FIGS. 4A-4D is a large diameter tube segment 401 and joined to inner surface of said element 401 may be a small tube element 402. On the proximal member 120, a marker band 406 may be applied, with a ball wire 403 extending proximally from marker band 406 and terminating at junction 150 (FIGS. 4A, 4B). The ball wire 403 may be passed through the small tube element 402 with the diameter of the ball wire 403 being slightly smaller than the inner diameter of the small tube element 402. Once the ball portion of ball wire 403 has fully passed through small tube element 402, a pull wire 405 can then be passed through small tube element 402 as in FIGS. 4C and 4D. The additive diameter of the pull wire 405 and the diameter of the ball wire 403, but not the ball itself, may not be greater than the inner diameter of the small tube element 402. The ball wire's 403 ball diameter plus the pull wire 405 diameter may exceed the inner diameter of the small tube 402, coupling the distal and proximal members 110, 120 until the pull wire 405 is removed. This pull wire 405 may be affixed to a region on the proximal hub 100a, allowing for removal by pulling it out of the device 100.

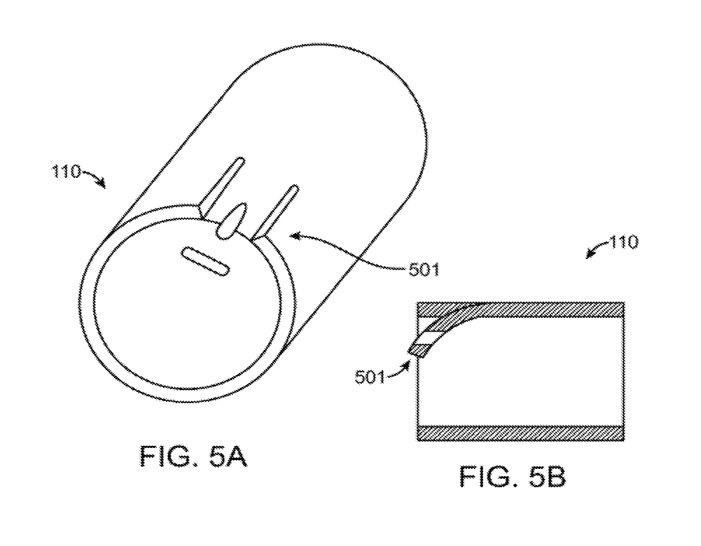

FIGS. 5A and 5B display proximal member 110 which may function in a similar fashion to the corresponding member 120 shown in FIGS. 4A-4D. The proximal member 110 may include a superelastic/shape memory element or tab 501 used to receive ball wire and pull wire components. This superelastic/shape memory element or tab 501 may function in a similar fashion to the small tube segment or element 402 shown in FIGS. 4A-4D. The tab 501 may replace the small tube segment or element 402 and the tab 501 may be integral to the proximal member 110. The tab 501 shown on the proximal member 110 may be heat set into a downward or upwards position to couple the distal member 120 and the superelastic properties of the tab 501 may enable it to relocate as an input is induced to the proximal hub 100a or interior lumen so as to allow a decoupling to occur. This tab 501, along with all other inner member components, may be situated anywhere along the entire length of the proximal member 110.

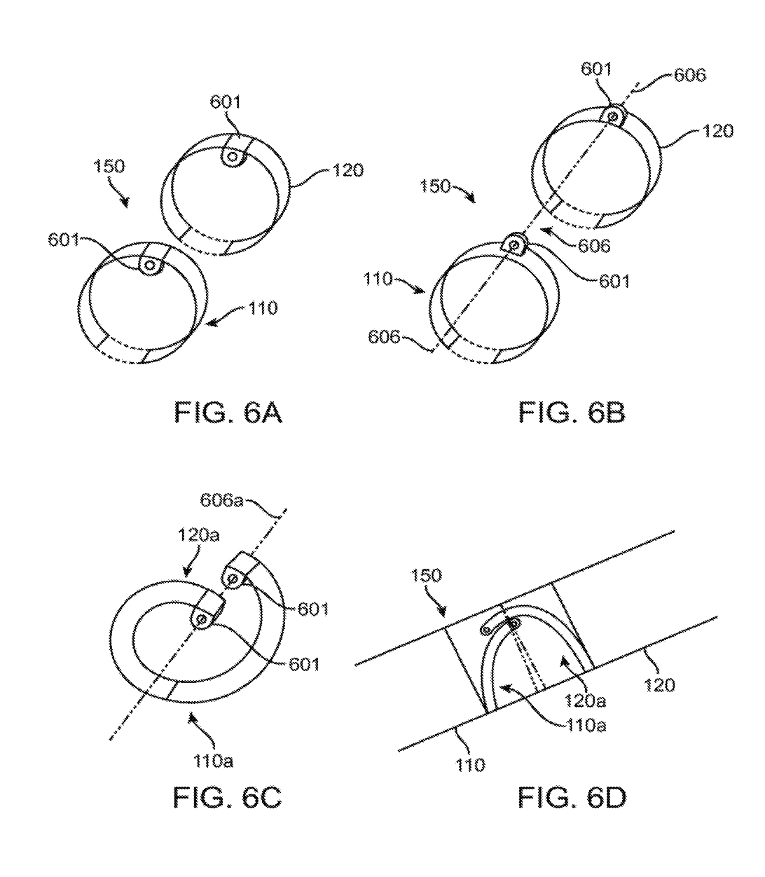

FIGS. 6A-6D show perspective views of the coupling region or junction 150 which utilizes locking ring elements 601 to join proximal and distal members 110, 120, which may be held coincident using a pull wire 606. FIG. 6A shows the proximal and distal members 110, 120 with locking ring elements 601 and FIG. 6B shows the same with the pull wire 606 threaded through the holes or apertures of the locking ring elements 601 to hold the proximal and distal members 110, 120 together. FIGS. 6C and 6D show locking ring elements 110a, 120b which may extend out from the interfacing ends of proximal and distal members 110, 120 at the junction 150. The two ring members 110a, 120a can be screwed (e.g., the ring members may have a high pitch such as a ¼ turn) and a wire 606a may lock the two ring members 110a, 120a in place. The two ring members 110a, 120a may be easily un-coupled once the wire 606a is removed. The locking ring elements 110a, 120a may have apertures or holes through which the pull wire 606a is threaded through.

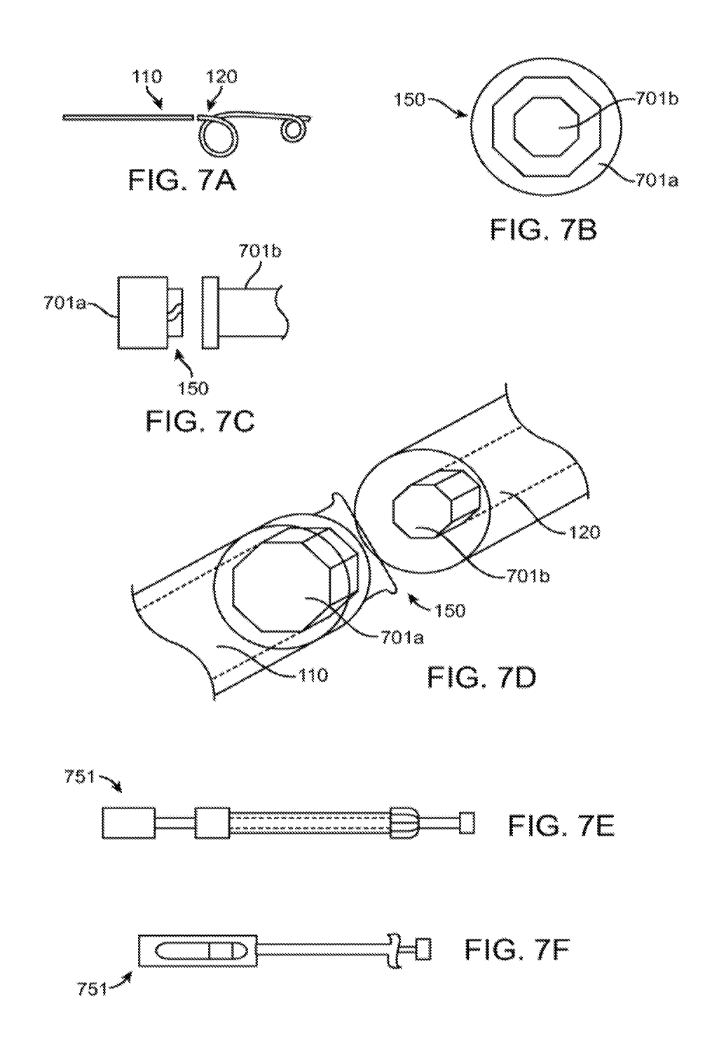

FIGS. 7A-7D show views of the coupling region or junction 150 which utilizes keyed (hexagonal shape shown to illustrate) elements affixed to proximal and distal members 110, 120 to achieve coupling. The inner member assembly coupling the stent to the catheter may be threaded/rotationally interlocked (like a ¼ turn or other thread) and keyed (e.g., hexed). As shown in FIG. 7A, one of the proximal or distal members 110, 120 may be held stationary while the other is turned to decouple the proximal and distal members 110, 120. A tool with coaxial members may be provided. The tool may hold one of the proximal or distal members 110, 120 stationary and can rotate the other to unscrew them. The tool may be put down the catheter/proximal member 110 (i.e., advanced within the lumen of the catheter) to engage the inner member assembly at the time of disconnect. The stent/distal member 120 may be held steady and the part affixing the stent/distal member 120 to the catheter/proximal member 110 may be unscrewed. The catheter/proximal member 110 may be twisted while the stent/distal member 120 is decoupled. A pull wire may not be necessary with use of the tool to assist decoupling. FIG. 7B shows a side section view of the junction 150, showing a first keyed portion 701a coupled to the second keyed portion 701b. FIG. 7C shows a side view of the junction 150, showing the first keyed portion 701a in alignment with the second keyed portion 701b. FIG. 7D shows a perspective section view of the same. While hexagonal shapes for the keyed portions are shown, other shapes such as star or torx like shapes may be used instead. The present disclosure also provides a counter rotating tool 751 to engage and unlock proximal and distal portions 110, 120, without twisting the distal portion 120 (FIGS. 7E, 7F). The handle mechanism of the tool may allow the keyed shape to be rotated while the outer bodies of the proximal and/or distal portions 110, 120 are held stationary.

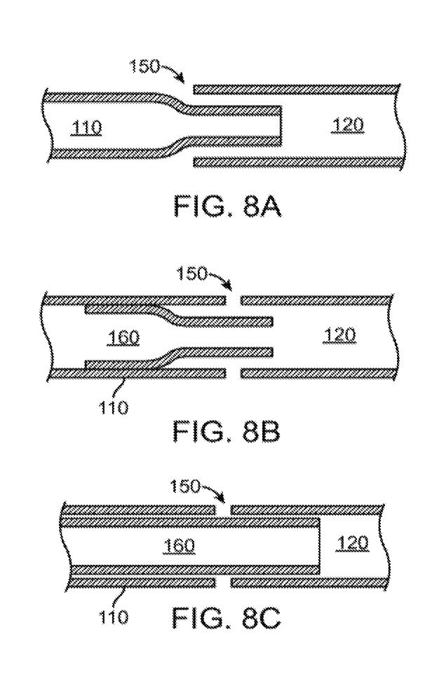

FIGS. 8A to 8B show side section views of the coupling region 150 in which the use of the inner member 160 of the catheter 100 is utilized in separate configurations to maintain concentricity between stent member 120 and catheter member 110. A variety of coupling mechanisms may be used with any of these inner member 160 configuration styles. FIG. 8A shows a configuration in which the functionality of the inner member 160 has been formed onto the distal tip of the catheter member 110. This distal tip of the catheter member 110 may slide into the lumen of the stent member 120. FIG. 8B shows a configuration whereby the inner member 160 does not extend back to the proximal hub 100a; this inner member 160 may be a component which is affixed to the catheter member 110 of the device 100 similar to the design style shown in FIGS. 4A-4D. FIG. 8C displays a configuration in which the inner member 160 extending fully back to the proximal hub 100a and being fixed into place at the hub region. The catheter distal tip may be formed to taper in toward the inside lumen of stent, it may utilize a fixed inner member at the catheter's distal end, or it may be a slidable component which may be fully removed from the catheter lumen.

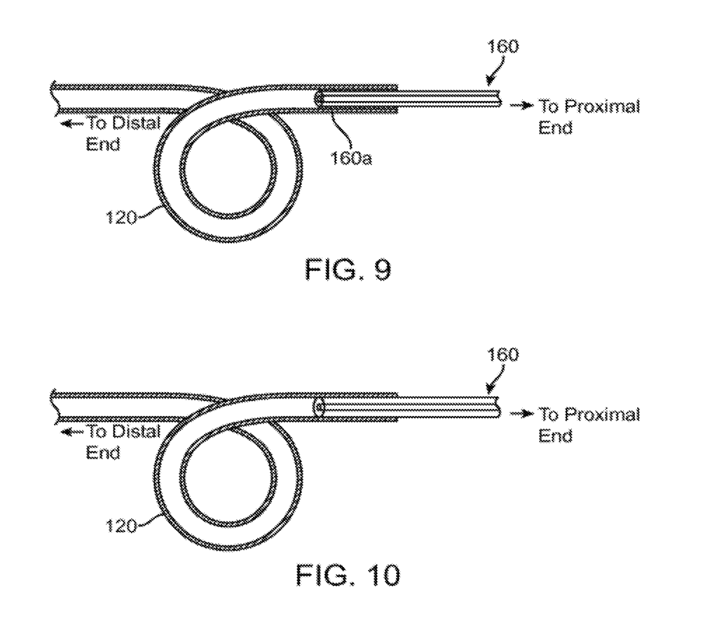

FIG. 9 shows the use of an adhesive may be applied to the region of the inner member 160 region 160a which is in contact with the lumen of the stent member 110. This adhesive joint may effectively couple the inner member 160 and its corresponding outer catheter member 110 to the stent member 120 (outer catheter member 110 not shown in FIG. 9 or 10).

FIG. 10 shows the use of an interference fit where the inner member 160 has an oversized diameter, which when passed into the lumen of the stent member 120, a frictional fitting joint is achieved.

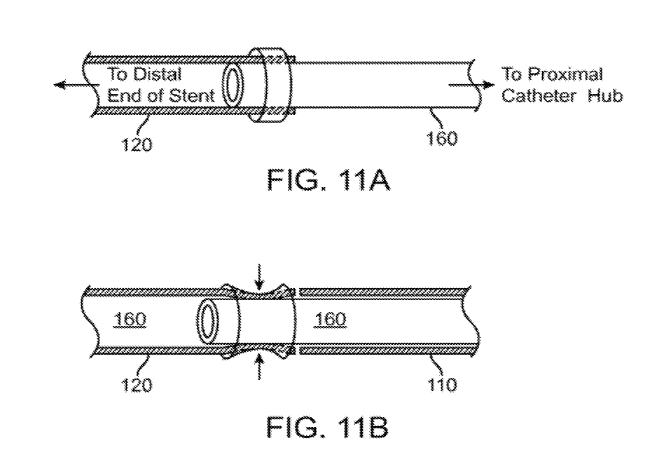

FIGS. 11A and 11B show the use of a metallic or polymeric crimp 1101 applied to the outside of the stent member 120, circumferentially collapsing that region over the inner member 160 of the catheter member 110 resulting in a coupled region.

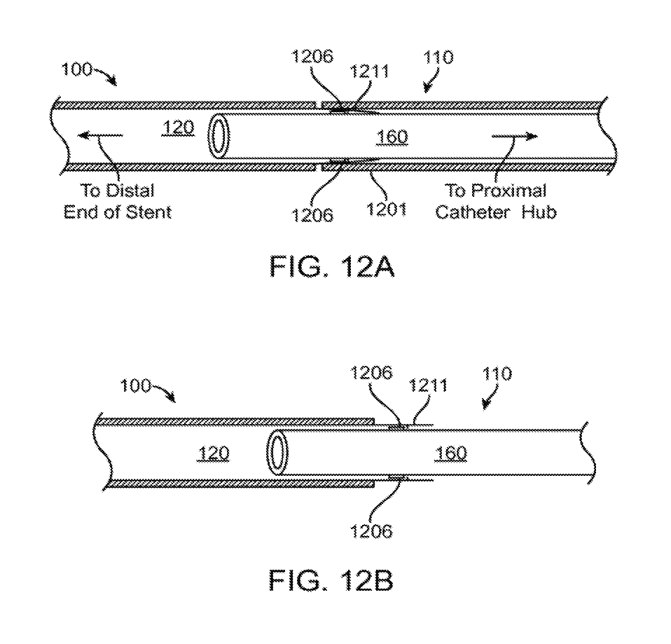

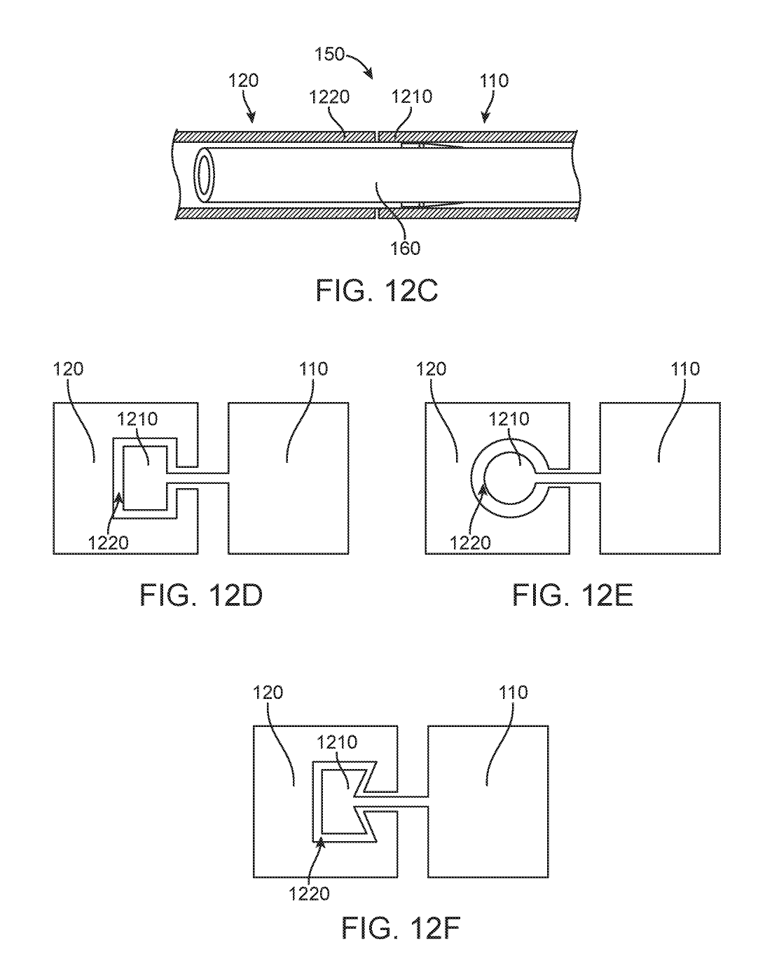

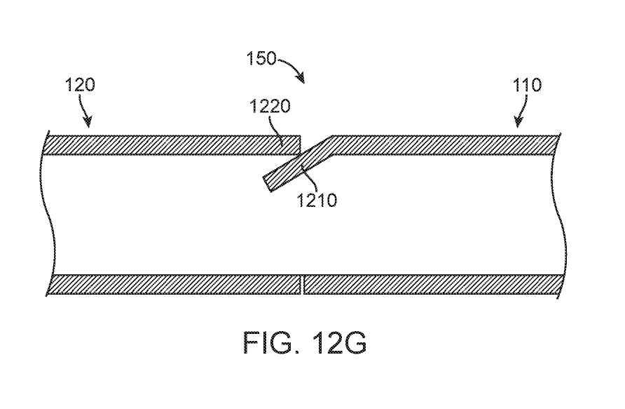

FIGS. 12A and 12B show the use of a superelastic/shape memory alloy which in this case has been affixed to the stent member 120 which may interface with protrusions 1206 affixed to the inner member 160 of the catheter 100. The stent member 120 may comprise a removable sheath 1201 acting as the catheter tube (FIG. 12A), whereby the removable sheath 1201 would be the first element of the device 100 to be removed resulting in the release (shape alloy memory effect) of the wires grabbing protrusions 1206 on the outer surface of the inner member 160 from a super elastic component 1211 of the stent member 120, thus allowing for the complete removal of the inner member 160 thereafter.

Referring to FIGS. 12C-12G, in some embodiments, a coupled junction 150 may be achieved with a flap 1210 on the catheter member 110, and a complementary shaped slot 1220 on the stent member 120. The slot 1220 will generally be shaped and sized to receive the flap 1210. An inner member 160 will typically traverse the junction 150 and the lumens of the catheter member 110 and stent member 120. When the junction 150 is coupled, the flap 1210 is located within the slot 1220 and the inner tube 160 crosses between the lumens of the catheter member 110 and stent member 110. As shown in FIGS. 12D-12F, separation of the catheter member 110 and the stent member 120 is restricted by the interaction between the flap 1210 and the complementary slot 1220. For instance, the proximal edges of the flap 1210 will abut the “arms” of the complimentary slot 1220 when the catheter member 110 and the stent member 120 are attempted to be pulled apart. FIG. 12F depicts an exemplary shape of the flap 1210 and exemplary shape of the slot 1220. FIG. 12F depicts a canting interlocking interface which can help prevent the catheter member 110 and stent member 120 from decoupling unintentionally when the catheter member 110 and stent member 120 are under tension. The flap 1210 will typically be biased radially inward toward the lumen of the catheter member 110, such that the flap 1210 may bend radially inward when no longer supported by the inner member 160 (which may be retracted from the junction 150) as shown in FIG. 12G, such that the flap 1210 is positioned out of the slot 1220 and the catheter member 110 and the stent member 120 may be separated.

FIG. 12C illustrates a side view of the coupled junction 150 wherein the flap 1210 abuts in the longitudinal direction a part of the complementary shaped slot 1220 and in the radial direction, the inner member 160 which keeps the flap 1210 flush with the body of the catheter 110. The abutments can prevent the catheter member 110 and stent member 120 from decoupling.

FIGS. 12D-12F show a top view of one embodiment of a coupled junction 150 by means of the flap 1210 and the slot 1220. As illustrated, the shape of the flap may take various forms. In some embodiments, the flap 1220 is shaped like a “T,” as shown in FIG. 12D. In other embodiments, as shown in FIG. 12E, the flap 1210 may take the shape of a neck with a circle or a sphere. In other embodiments, as shown in FIG. 12F, the flap 1210 may take the shape of a neck with a chevron-shaped body to form the canting interlocking interface. Further, while only one assembly of the flap 1210 and the slot 1220 is shown, the catheter 110 and the slot 120 may have more than one flap 1210 and slot 1220, such as a pair of the flap 1210 and the slot 1220 that are diametrically opposed to even out the longitudinal separation force throughout the junction 150.

As shown in FIG. 12G, when the inner member 160 is retracted, the flap 1210 no longer abuts the inner member 160, returns to its natural bent shape, and is removed from the slot 1220, thereby uncoupling the catheter member 110 and the stent member 110.

In some embodiments, the coupled junction 150 may be provided by the flap 1210 being on the stent member 120 instead of the catheter member 110, and with the slot 1220 being on the catheter member 110 instead of the stent member 120. In some embodiments, the stent member 120 may include both the flap 1210 and the slot 1220, which are diametrically opposed to one another, and the catheter member may include a complementary set of the flap 1210 and the slot 1220 such that the junction 150 can be formed. While a single flap and a single complimentary slot are shown, multiple flaps and complimentary slots may be used. While the catheter member is shown as having the flap and the stent member is shown as having the complimentary slot, the attachment mechanism may be reversed, with the stent member having the flap and the catheter member having the complimentary slot. In some embodiments, the catheter and stent members may have multiple flap and complimentary slot mechanisms, with one or more being in the reserve orientation.

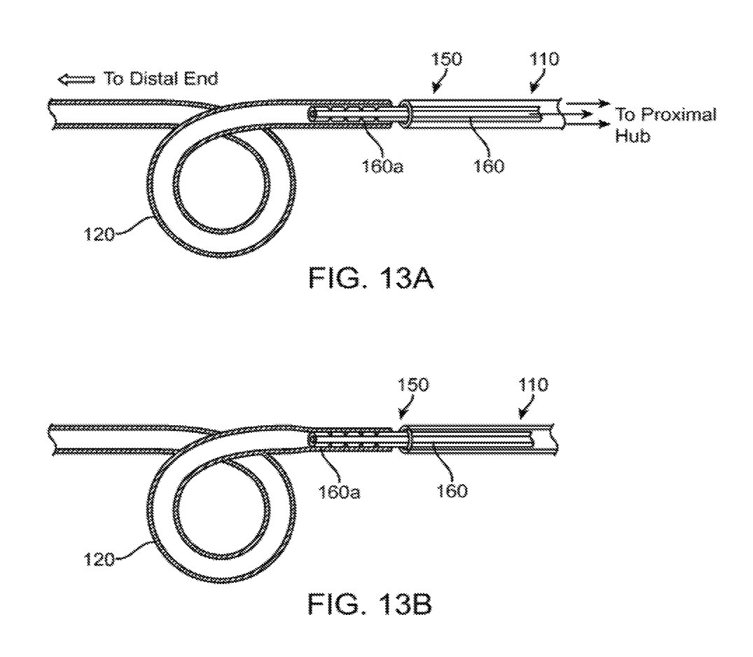

FIGS. 13A and 13B show where the region of the stent member 120 that interfaces with the inner member 160 is thermally processed. FIG. 13A shows the coupling region or junction 150 before processing and FIG. 13B shows the coupling region or junction 150 after processing. The inner member 160 may have a series of grooves 160a cut circumferentially about its surface, these grooves 160a serving as a region with which the polymer material of the stent member 120 may flow into when said region is heated. Once the stent member polymer is heated and has joined to the inner member 160, it is then allowed to cool permanently forming a mechanical interface between the two elements. A light to moderate pull force applied to the inner member 160 would allow it to break away from the stent member 120.

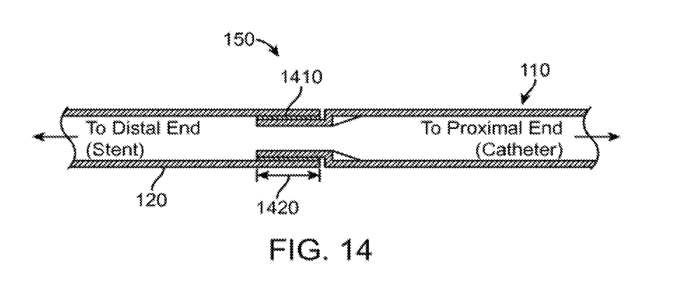

FIG. 14 details a coupling configuration which permits the distal member (stent) 120 to receive a threaded proximal member (catheter) 110 into the lumen of the stent member 120. The female threaded coupling region 1420 of the stent member 120 may receive a proximal member 110 which has a male type thread arrangement 1410 over its outer surface within its coupling region 150. This permits proximal member 110 to be coupled to distal member 120 by threading into position and later proximal member 110 may be removed by unthreading (rotating) the proximal portion 110 of the device 100.

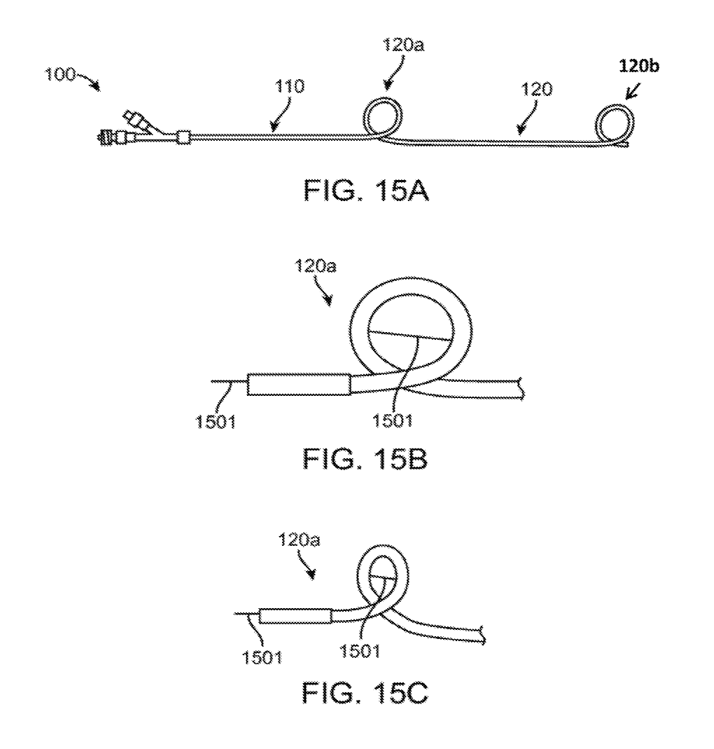

FIGS. 15A-15C depict the strand material used to close the proximal loop 120a of the distal portion (stent) 120 of the device 100 once it is within the renal pelvis of the kidney. This strand of material referred to as the proximal suture loop 1501 may pass through holes located through the sidewall of the bottom of the loop 120a. As the distal stent loop or curl 120b and the proximal stent loop or curl 120a are straightened for delivery, the distal curl 120b of the stent member 120 may reform upon straightener removal due to the large space in the bladder. The proximal loop 120a may need mechanical encouragement to reform in the tighter renal pelvis region. The present device 100 can use a proximal loop suture 1501 which is pulled in tension at the proximal hub 100a of the device 100 to reform the proximal loop 120a of the stent 120. This proximal loop suture 1501 may be removed by cutting one end of the strand at the hub 100a and pulling on the other end until it is fully removed. The proximal suture loop 1501 and its function may act independently of the coupling mechanism. Appropriate materials for the proximal suture loop 1501 may be UHMWPE (ultra-high molecular weight polyethylene), metals, fluoro-polymers, PEEK, HDPE, liquid crystal polymers (e.g., Vectran), and combinations thereof.

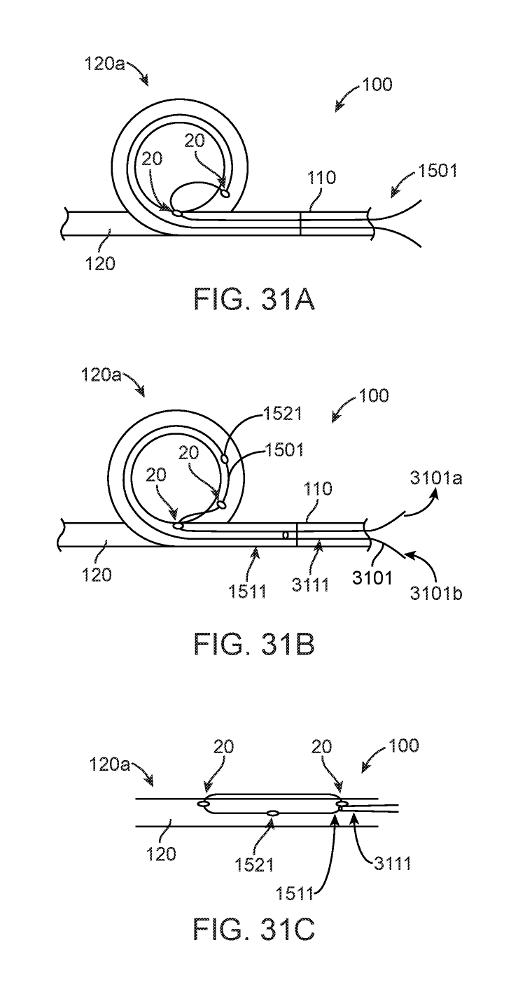

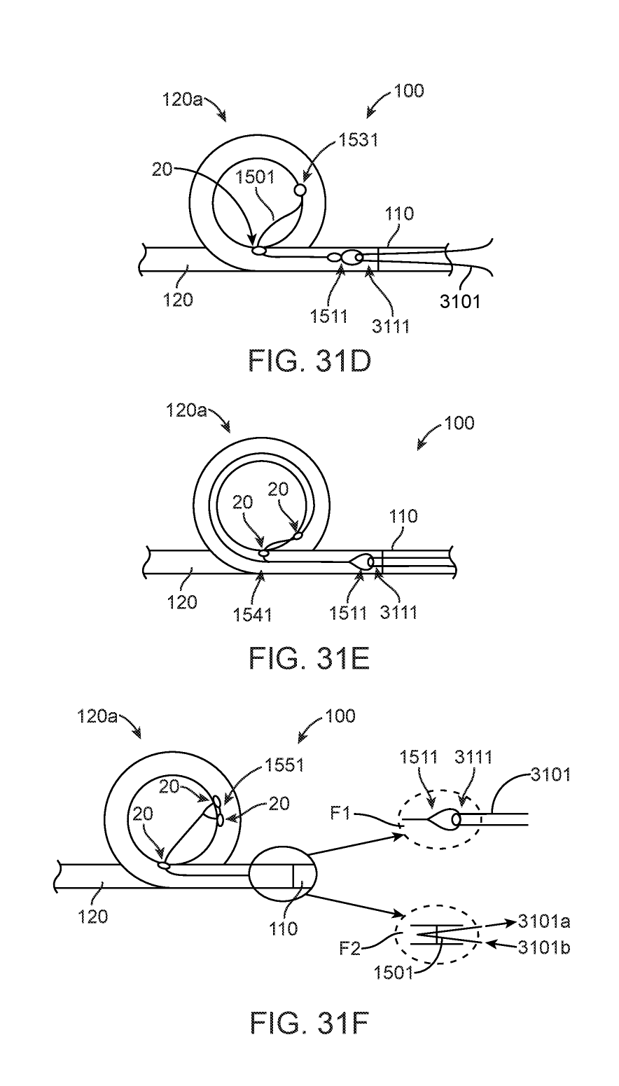

Referring to FIGS. 31A-31F, further loop forming and straightening mechanisms are described.

As shown in FIG. 31A, the proximal loop suture 1501 may distally traverse through the catheter member 110 and the stent member 120 before exiting from one of the exit holes 20 of the stent member 120. The proximal loop suture 1501 may then re-entering the stent member 120 through another, more distal exit hole 20, before proximally traversing through the stent member 120 and the catheter member 110. Tensioning the proximal loop suture 1501 such as by pulling the proximal loop suture 1501 from the proximal hub 100a can cause the portion of the proximal loop suture 1501 traversing the proximal loop 120a to shorten and thereby form a loop. As shown in FIG. 31A, at least two strands of the proximal loop suture 1501 may reside within the catheter member 110 and one or more of these two strands may be pulled. In some embodiments, tensioning the proximal loop suture 1501 may apply an inward force and/or friction to the exit holes 20. In some embodiments, the exit holes 20 may be reinforced such as with additional material or an O-ring.

As shown in FIG. 31B, the proximal loop suture 1501 may form a loop and be enclosed entirely within the proximal loop 120a. The proximal most portion of the proximal loop suture 1501 may form an end loop 1511. One strand of the proximal loop suture 1501 may exit from one of the exit holes 20 of the stent member 120 before re-entering the stent member 120 through another, more distal exit hole 20. The other strand of the proximal loop suture 1501 traverses the stent member to meet the first strand, and the ends of the proximal loop suture 1501 may be tied together within the stent member 120 to form a knot 1521 which closes the loop. A further pull suture 3101 is provided and pull suture 3101 traverses the catheter member 110 and forms an end loop 3111 which interlocks with the end loop 1511 of the proximal loop suture 1501. The two proximally facing strands of the pull suture 3101 may be pulled to tension the proximal loop suture 1501 and form the proximal loop 120a into a loop. The pull suture 3101 may be released from tension to allow the proximal loop 120a to straighten as shown in FIG. 31C. The pull suture 3101 may be released from interlocking the end loop 3111 of the proximal loop suture 1501 by freeing one strand of the pull suture 3101 and pulling the other so that eventually the free end passes out of the end loop 3111. For example, one strand of the pull suture 3101 may be pulled in a proximal direction 3101a and the other may advance distally in a direction 3101b before the end of the pull suture 3101 passes through the end loop 3111 before reversing direction to be pulled out.

As shown in FIG. 31D, instead of being in the form of a loop with two strands passing through the proximal loop 120a as shown in FIGS. 31B and 31C, the proximal loop suture 1501 may be in the form of a single strand with one end coupled (for example, glued or tied into) the end loop or ring 1511 and the other end passing though the exit hole 20 before being fixedly coupled to (for example, glued or tied into) fixation location 1531 on stent member 120. The proximal loop suture 1501 may be tensioned and released with the pull suture 3101 as described with respect to FIGS. 31B and 31C.

As shown in FIG. 31E, instead of having one end of the proximal loop suture 1501 being fixedly coupled to the stent member 120a as shown in FIG. 31D, the two ends of the proximal loop suture 1501 may be tied into the end loop 1511 to the end loop 3111 of the pull suture 3101 and another end loop 1541. Distally of the end loop 1511, the proximal loop suture 1501 traverses the proximal loop 120a, exits the more distal exit hole 20 on the stent member 120a, and re-enters the stent member at the more proximal exit hole 20 to loop around the proximal loop suture 1501. Tensioning or pulling the proximal loop suture 1501 from the end loop 1511 can collapse the proximal loop 120a by a “noose-like” action. The proximal loop suture 1501 may be tensioned and released with the pull suture 3101 as described with respect to FIGS. 31B to 31D.

As shown in FIG. 31F, instead of having one end of the proximal loop suture 1501 being fixedly coupled to the stent member 120a as shown in FIG. 31D, that end of the proximal loop suture 1501 may instead form a loop or ring 1551 passing through a pair of end holes 20 in the stent member 120. The other end of the proximal loop suture 1501 passes through a more proximal exit hole 20 on the stent member 120 before traversing proximally from the stent member 120 to the catheter member 110. This other end may be pulled proximally to tension the proximal loop suture 1501 to cause the proximal loop 120a to from a loop. This other end may form an end loop or ring 1511 to be tensioned by separate pull suture 3101 as in FIGS. 31D and 31E (as shown with magnified area F1 in FIG. 31F) or may be manipulated directly as in FIG. 31A and/or in a similar manner as shown in FIG. 31B (as shown with magnified area F2 in FIG. 31F).

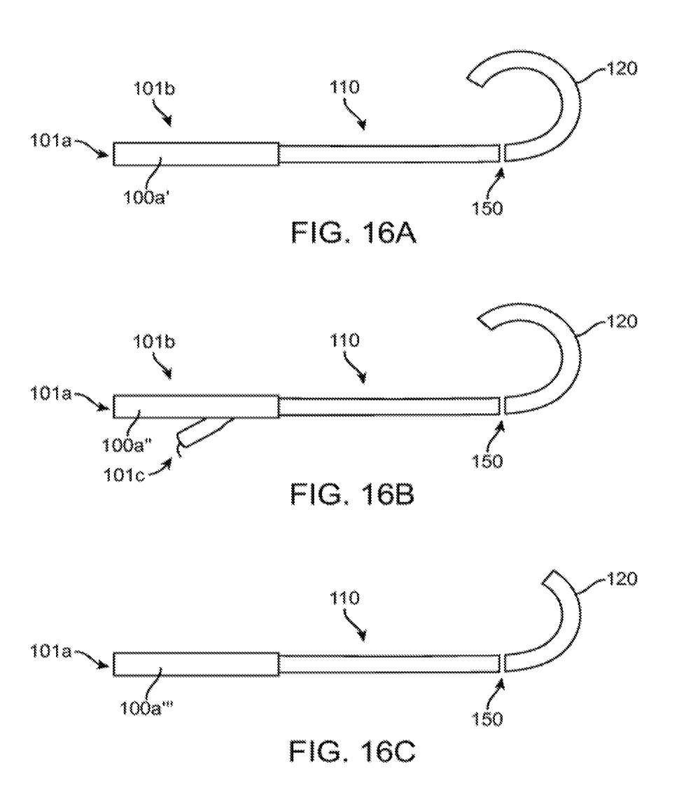

FIGS. 16A, 16B, and 16C display several configurations of proximal hubs. The coupling components may extend out from various hub configurations enabling removal and/or features such as a push button may permit decoupling of the device. That is, the pull wire(s) or suture loop(s) may be retracted from various ports of the proximal hubs. FIG. 16A shows a proximal hub 100a′ with a main port 101a and a lateral port 101b. In an example, the suture loop 1501 may be proximally retracted from the lateral port 101b to facilitate the (re)formation of the proximal loop 120a and the pull wire 140 may be retracted from the main port 101a to release the stent member 120. FIG. 16B shows a proximal hub 100a″ with a main port 101a and two lateral ports 101b, 101c. The additional lateral port 101c may, for example, be used for retraction of the suture 130 after the pull wire 140 has been retracted. FIG. 16C shows a proximal hub 100a′″ with only a main port, which may be used for one or more of the pull wire(s) or suture(s). When the stent member 120 has been left implanted in the patient, the suture(s) may be one or more of cut, retracted, or left in place.

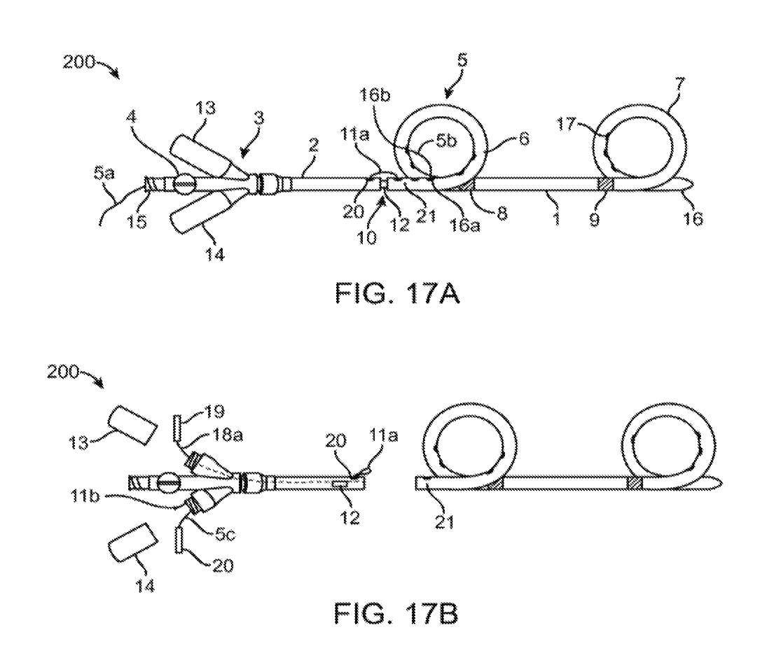

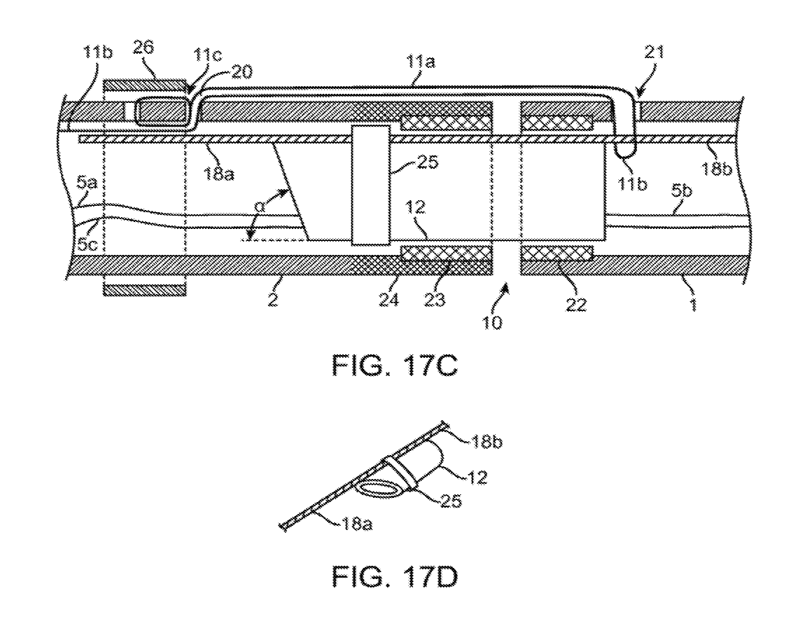

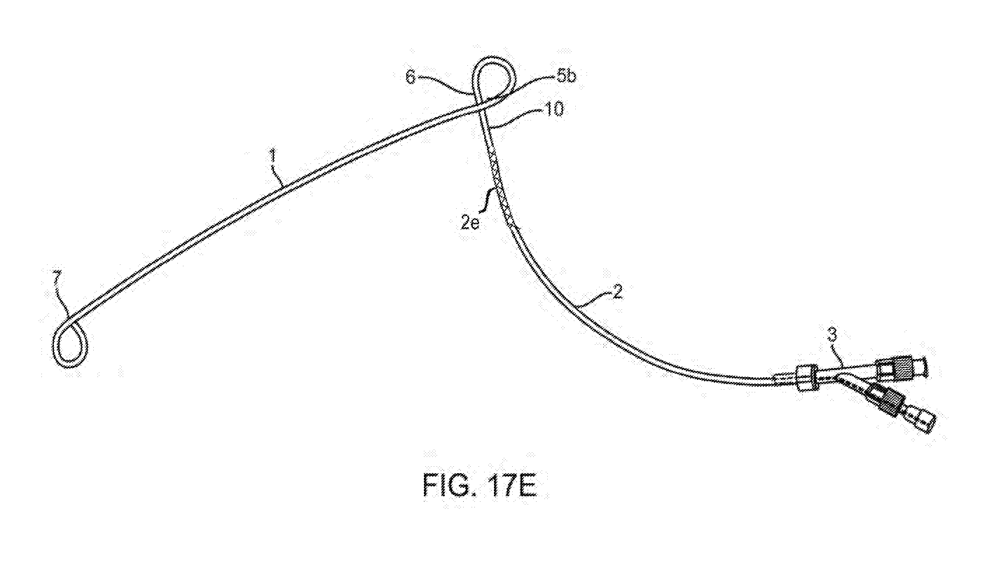

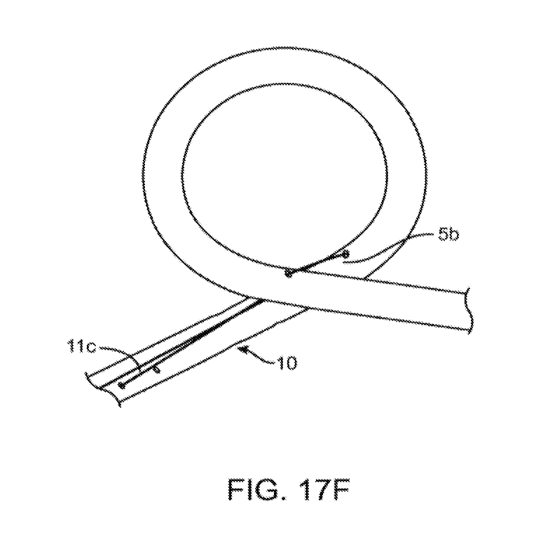

U.S. Pat. Nos. 8,657,884 and 9,387,312, which described convertible stents and catheters similar to those described herein, are each incorporated herein by reference. Referring to FIGS. 17A-17F, in some embodiments, the catheter member 2 and the distal member 1 may comprise a tube that is flexible. The material of the tube may be UHW polyethylene, PTFE, or any other suitable material known in the art. The tube may have a plurality of holes that extend through the tube so that fluids may flow into or out of the tube through the holes. In some embodiments, the tube is of sufficient length so that it extends from the outside of the patient into the kidney, through the ureter and into the bladder. The length of the tube may vary according to the application of the system 200.

Referring to FIGS. 17A-17B, the distal stent member 1 may comprise two loops 6, 7. The distance between the two loops 6, 7 may vary depending on the application of the system 200. For example, for uretheral applications, the distance between the two loops 6, 7 may be between 25 and 35 cm, such as between 27 cm and 32 cm, such as 28 cm. As another example, for biliary applications, the distance between the two loops 6, 7 may be between 5 and 10 cm, such as 7 cm.

In some embodiments, the diameter of the lumen of the tube may be 10F. The diameter of the lumen may remain constant throughout the length of the lumen. In other embodiments, the diameter of the lumen can vary throughout the length of the lumen. For example, the diameter of the lumen may taper from the proximal end to the distal end. In some embodiments, referring to FIG. 17A, the lumen of the proximal catheter member 2 can have a diameter of 10F, the lumen of the proximal loop 6 can have a diameter of 9F, and the lumen of the distal loop 7 can have a diameter of 8F. The diameters of the various sections of the lumens can be adjusted according to the desired application. For example, the tapering may be provided to ease advancement of the convertible catheter/stent.

In some embodiments, the method of using the device comprises advancing the device through a percutaneous access site so that the distal end of the catheter portion of the device is positioned in at or near a first bodily lumen, such as a bladder, with the proximal end of the stent member 120 being positioned in the first bodily lumen. In some embodiments, the catheter member 110 is left in the patient's body for a few days before the catheter member 110 is removed. In many cases, it can be desirable to achieve hemostasis at the tissue area where the catheter member 110 is placed. In some instances, hemostasis can be achieved at this tissue area by coating the catheter member 110 with hemostatic coating agents (for example, a hemostatic element 2e at the proximal catheter member 2 as shown in FIG. 17e). Examples of hemostatic coating agents include microfibrillar collagen hemostat, chitosan, kaolin, zeolite, styptics, anhydrous aluminum sulfate, any other suitable hemostatic coating agent, or any combination thereof. In other embodiments, hemostasis can be achieved through electrocautery (for example, the hemostatic element 2e at the proximal catheter member 2 as shown in FIG. 17e). For instance, the catheter portion of the device may include one or more electrodes to ablate the surrounding tissue and arrest blood flow. When hemostasis is achieved early, the catheter member 100 may be removed early as well.

Further nephrouretheral stent systems and joining or coupling mechanisms are described below. Many of the elements of the figures and their corresponding reference numbers are listed below.

1: Stent

2: Detachable drainage/delivery catheter

3: Hub

4: Loop suture lock

5: Loop suture
5a: Tensioning end of loop suture
5b: Loop locking end of loop suture
5c: Removal end w/tab (for lock suture proximal exit hole 20)

6: Proximal loop

7: Distal loop

8: Distal radiopaque marker

9: Proximal radiopaque marker

10: Junction stent to drainage catheter (shown with gap for clarity)

11: Lock Suture
11a: Distal lock suture loop
11b: Hub attachment (example of possible location)
11c: Distal lock suture tie down

12: Coupler, retractable

13: Protective cap (pull wire)

14: Protective cap (for loop suture 5)

15: Lure thread connector (standard)

16: Tapered tip
16a: Drainage hole
16b: Drainage hole

17: Drainage holes (interior of loops)

18: Lock/Release wire
18a: Proximal part (going from coupler 12 to pull tab 19)
18b: Distal end (going through lock distal lock suture loop 11a)

19: Lock/Release wire pull tab

20: Lock suture proximal exit hole

21: Lock Suture Distal entry hole

22: Distal reinforcement on stent (e.g., SS hypotube)

23: Proximal reinforcement on catheter (e.g., SS hypotube)

24: Alternative reinforcement or in combination with other reinforcement, higher durometer or tougher tubing than main body

25: Advancement Stop

26: Lock suture tie down reinforcement (swaged hypotube, for example, not shown swaged flush for clarity)

27: Separate lock/release wire

28: Inner member

29: Fixed coupler

30: Coupler to catheter attachment

31: Slip fit

32: Lock wire

33: Wire

As shown in FIGS. 17A-17F, a nephrouretheral stent system 200 may comprise three major components: a distal and releasable stent or stent member 1, a catheter 2 and a hub 3, and a coupling and release mechanism which may comprise a loop suture 5, a lock suture 11, a retractable coupler 12, a lock/release wire 18, and a lock/release wire pull tab 19. The hub 3 may be fixed to the catheter 2, while the stent 1 may be releasably fixed to the catheter 2 at the junction 10 by the coupling and release mechanism. The coupling and release mechanism may operate in a manner similar to the coupling mechanisms described above and herein. For example, referring to FIG. 17C, the lock/release wire 18 may threaded through the distal lock suture hub attachment 11b of the lock suture 11, the lock/release wire 18 may be retracted therefrom to release the lock suture 11 such that the stent 1 may decouple from the catheter 2, and the lock suture 11 may be proximally retracted further.

A straightener (e.g., a hypotube with a hub) can be put in to straighten the loops 6, 7 of the stent 1 out and the system 200 can be put over a guidewire in the body to be placed. The straightener can be then removed allowing the proximal and distal loops 6, 7 of the stent 1 to form. Usually, the proximal loop 6 will not form on its own in tight spaces and may need to be formed by pulling on the loop suture 5 similarly described above with reference to FIGS. 15a-15c.

As shown in FIGS. 17A-17F, one end of the loop suture 5c may be tied down to a pull tab 20, the loop suture going down the inner lumens of the catheter 2 and of the stent 1 to the proximal loop 6 where it may exit one drain hole 16a and re-enters another drain hole 16b and returns to the hub through the loop suture lock 4. The two drainage holes 16a, 16b may be configured so that when the loop suture 5 is tensioned, such as by pulling on tensioning end 5a, the loops suture 5 pulls the proximal loop 6 into a loop. The loop suture 5 can be locked in place by lock mechanism 4 to help retain the system 200 in the body. Additional drainage holes may exist on the proximal loop 6, generally residing on the inner portion of the loop 6.

In some embodiments, the nephrouretheral systems may not need the loop suture 5 removed. In such systems, the loop suture lock 4 can be unlocked to free up the proximal loop 6 and the whole catheter 2 including the loop suture mechanism can be removed. Such systems may not require the distal lock suture tie down 5c and the lock suture proximal exit home 20; and instead, the ends of the loop suture 5 may be un-accessibly tied down in the hub 3. Nevertheless, it can be critical to be able to withdraw the loop suture 5 entirely before converting and releasing the stent 1. Hence, the distal lock suture tie down 5c and the lock suture proximal exit home 20 can be accessible.

The tension in the loop suture 5 can be relieved by unlocking the loop suture lock 4, which can allow the proximal loop 6 to relax and un-fold as the system 200 is removed through an access channel/hole.

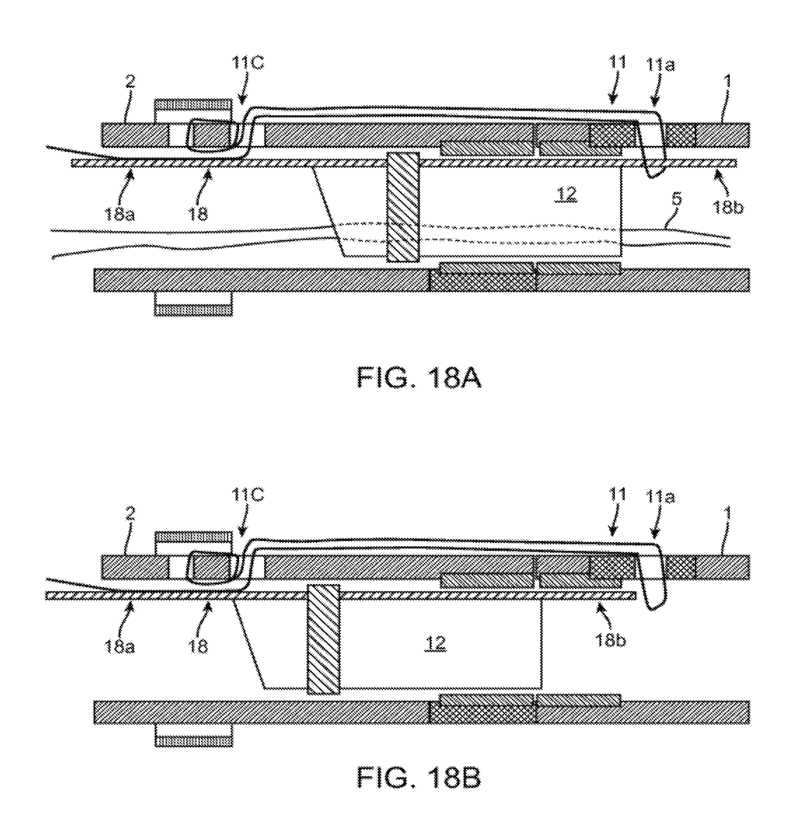

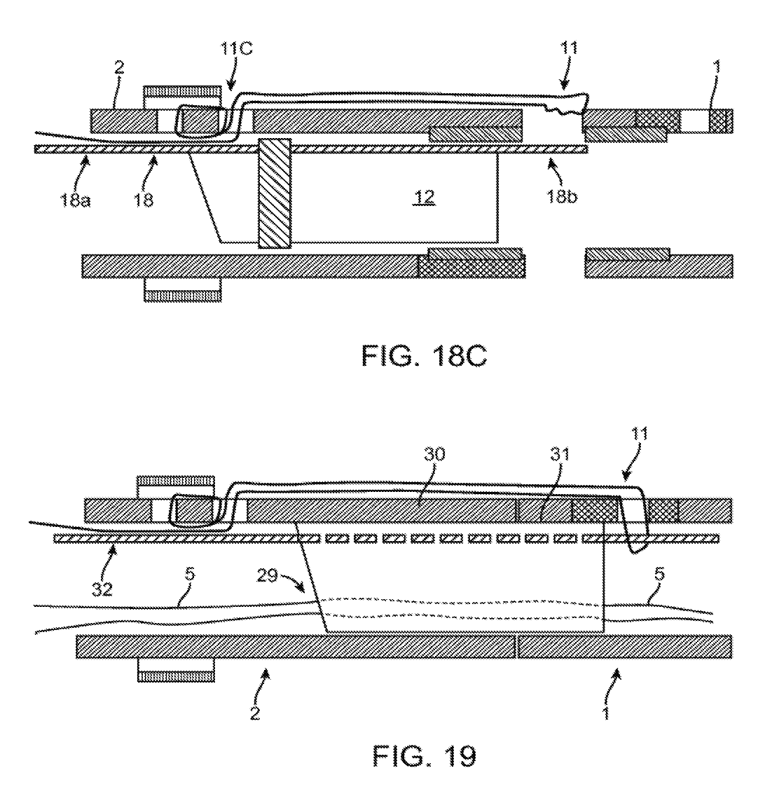

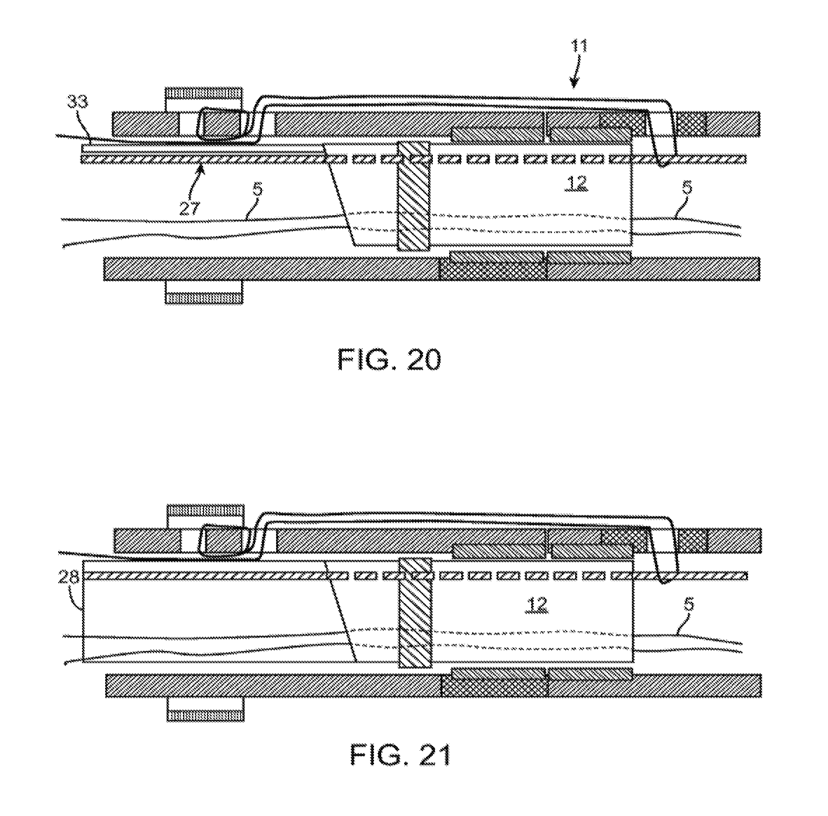

Referring to FIGS. 18A-18C, the coupling or lock mechanism for the system 200 can be similar to those described above and herein. The lock mechanism may comprise a lock wire (pull wire) 18 that may be permanently affixed to a coupler (e.g., a coupling cylinder) 12 of the inner member and may pass beyond the coupling cylinder 12 to engage or thread through the lock suture 11 at the distal lock suture tie down 11c as shown in FIG. 18A. As shown in FIG. 18B, the lock wire 18 may be retracted to free the distal lock suture tie down 11c. Such retraction frees the lock suture 11 and can retract the coupler 12 from the stent 1, allowing the lock suture 11 to be retracted and the stent 1 to be released.

In some embodiments, a coupler cylinder 29 may be affixed to the catheter 2 through the coupler to catheter attachment 30 and may not be able to be independently pulled back (FIG. 19). A lock wire 32 retractable to free the lock suture 11 may be separate from the coupler 29. In some cases, however, the fixed coupler 29 may hang up on inside the stent 1 during removal (for example, due to friction, biofouling, etc.) if not pulled back independently.

In some embodiments, the coupler 12 may be connected to a wire 33 that is separate from the lock/release wire 27, and the wire 33 may be pulled as an additional step (which could be mitigated by interlocking the pullback actions).

In some embodiments, the coupler 12 may be attached to a co-axial inner member 28, which may be affixed to the hub so that it can be pulled back. The coupler 12 may comprise an inner member, which may be solid polymer, nitinol, braided or coiled shafts (not shown).

Referring back to FIGS. 17A-17F, to deploy the stent portion 1, the operator may first unlock the loop suture 5, remove the loop suture cap 14, and pull the loop suture out. To actually deploy the stent 1, the cap 13 may be removed and the lock wire 18 may be pulled back by pulling the lock wire tab, which may comprise the cap 13. The lock wire 18 may be in communication (e.g., attached) to the coupler 12 such that the coupler 12 may be pulled back while it is pulling out of the lock suture loop 11b, disconnecting the stent 1.

As shown in FIG. 17C, an exemplary method of fixing the lock suture 11 is for one end 11c to be fixed near the distal end of the catheter 2 and the other end 11b fixed to the hub 3 for tensioning the catheter 2 and stent 1 together after the lock wire 18 is in place. Fixing one end 11c near the distal end of the catheter 2 while having the other end 11b be fixed more proximally can reduce instances of the catheter material pulling back or bunching up (and gaping at the junction) as the system 200 is advanced. As shown in FIG. 17C, the tie down end 11c may be tied down and fixed around a pair of holes on the outer wall of the catheter 2. Alternatively, the tie down end 11c may be tied down around the proximal part 18a of the lock wire 18. Alternatively, both ends of the lock suture 11 can be tied down at the distal end or fixed down at the hub 3. The lock suture ends may be locked down or fixed by tying around two holes, gluing, embedding, swaging marker, etc.

FIGS. 17A-17F depict embodiments where the loop structure 11 is on the same side as the proximal loop 6 and distal loop 7. In some embodiments, the loop structure 11 may be placed 180° around the circumference of the system 200 such that the loop structure 11 is on the opposite or bottom side of the proximal and distal loops 6, 7. In these embodiments, the mechanisms described above for tensioning the loop structure 5, locking the loop structure 5, removing the loop structure 5, relieving the tension in the loop structure 5, and deploying the stent portion 1, may be adapted accordingly. Orienting the loop structure 11 on the opposite side as the proximal and distal loops 6, 7, may reduce the risk of the suture tearing and catching with the internal cylinder.

The lock suture 11 may be made of a high tensile strength, low elongation material and flexible material like UHMW PE (Spectra, Honeywell) or other material, including stainless steel or other metallic materials, or a combination of materials. It could be a single ribbon with a hole at the end to pass lock wire through, or other configurations.

The stiff coupler 12 may be made of implant grade materials such as stainless steel, NiTi, PEEK, or other materials know in the art. More flexible couplers are possible, but do not support the catheter 2 and stent 1 at the junction 10 under bending, resulting in splaying open of the junction 10.

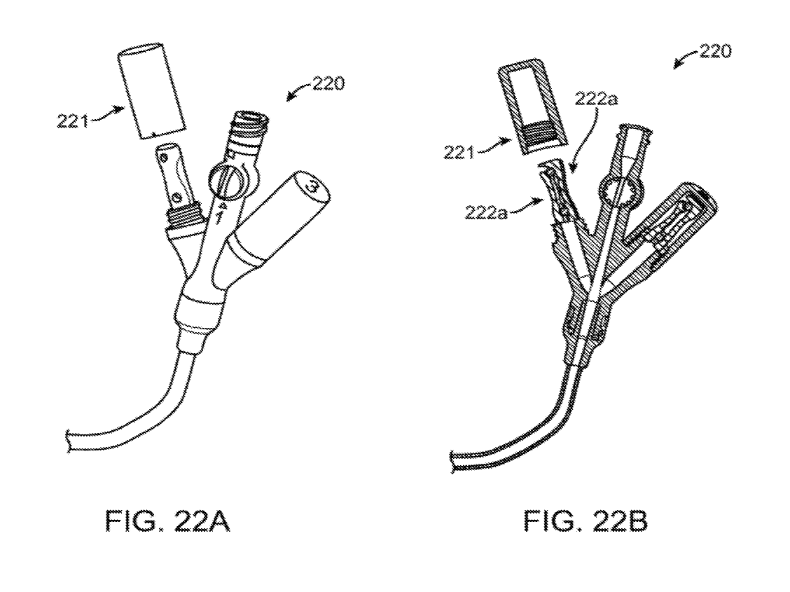

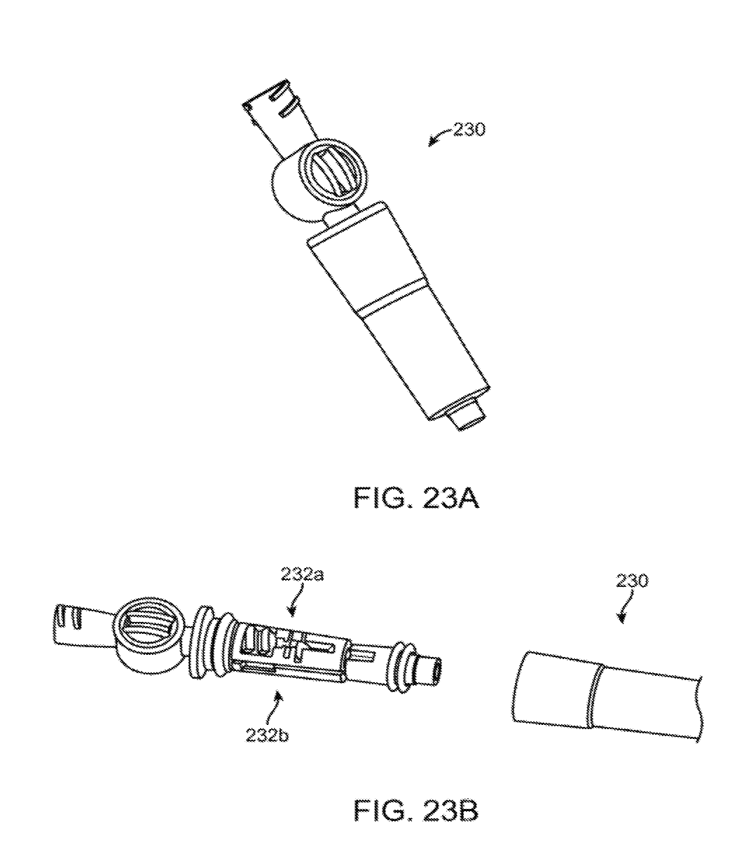

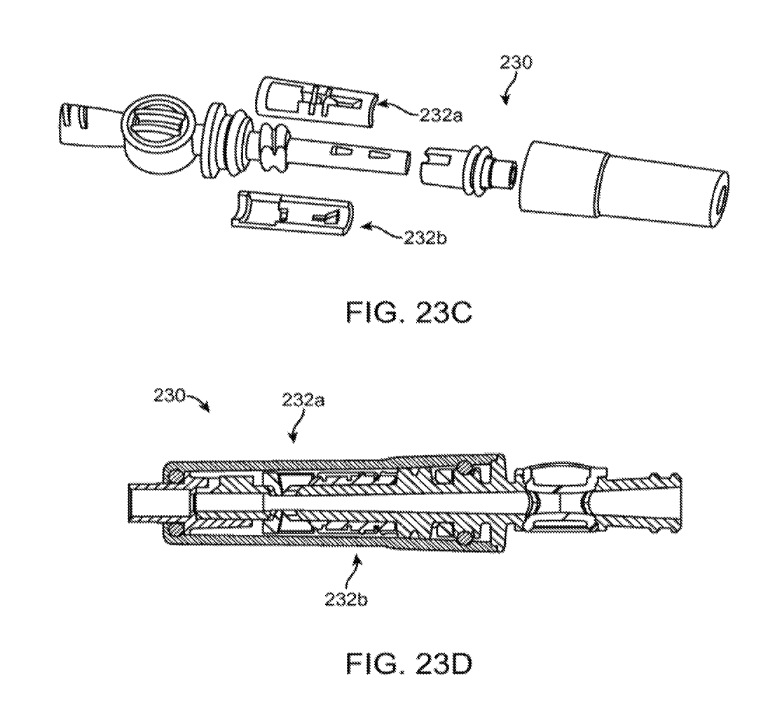

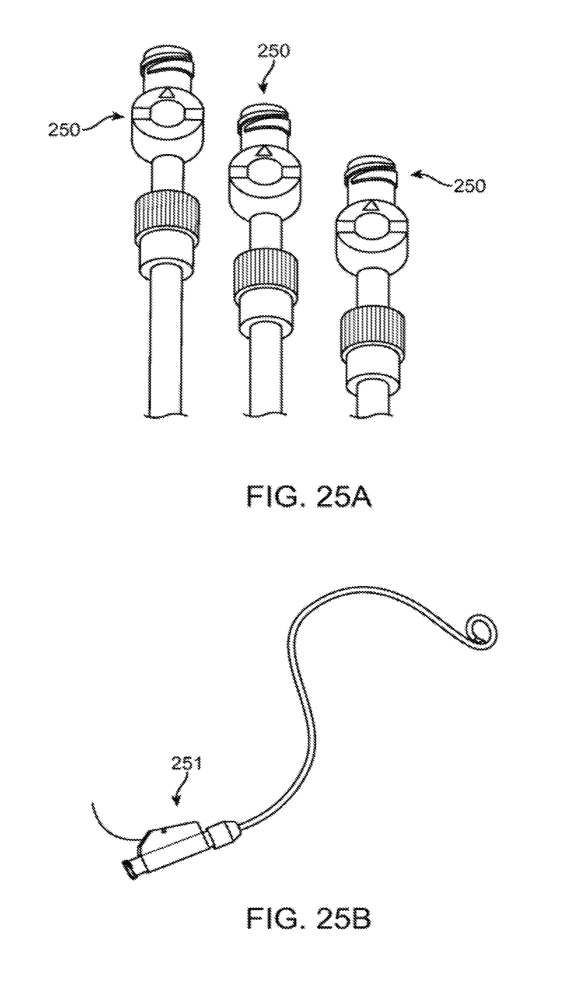

Various configurations of hubs are also disclosed, including a triple arm hub 220 which may be preferred in at least some cases (FIGS. 22A, 22B). A single side arm 221 of the triple arm hub 220 may comprise two pull tabs 222a, 22b. The hubs can be in axial configurations with pull tabs or laid out in a side arm or triple arm configuration. Alternatively, the hubs 220 could be axially stacked components (like rocket stages), that separate (unscrew for instance) in sequence to provide the necessary actions (FIGS. 23A, 23B, 23C). As shown in FIGS. 23B and 23C, the body of the hub 230 may be axially pulled apart so that pull tabs 232a, 232b may be accessed. A handle with a slide or twist mechanism may be used in some embodiments. The hubs shown may use a rotating hemostat type valve (shown in FIG. 25A by locks 250, for example) to wrap and lock the loop suture 5, although other mechanisms such as a center lever to lock the suture and seal out the side (shown in FIG. 25B, by lever mechanism 251, for example) may be used as well.

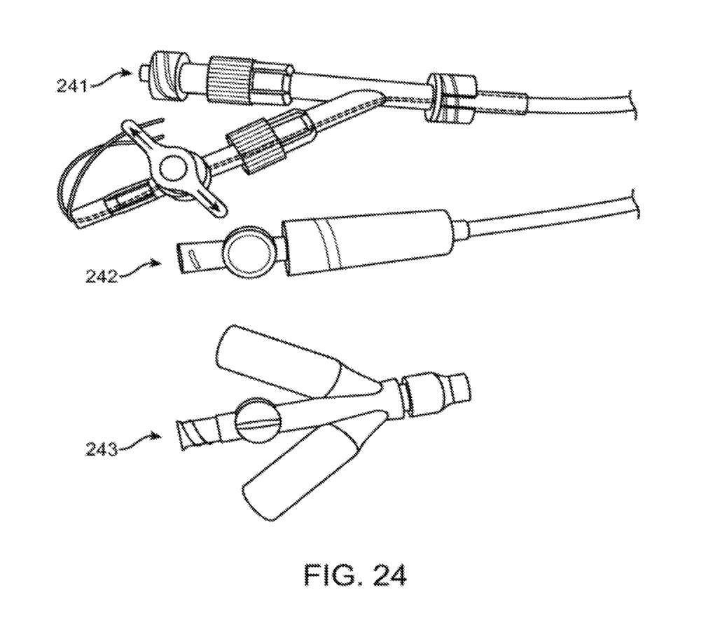

FIG. 24 shows further hubs that may be used for the devices 200, include a side armed hub 241, a barrel hub 242, and a triple armed hub 243.

In the side or triple arm hubs described above, the wire or sutures could be affixed directly to the caps, but may twist and bind if not provided a anti twist feature in cap. Since ports 15 on these devices 200 may need to be flushed periodically, a person un-familiar with the devices 200 might unscrew a cap inadvertently. Hence in preferred embodiments, pull tabs are separate from caps.

In some embodiments, the catheter 2 and the stent 1 may be decoupled from one another electrolytically or by electrical resistance based melting of a connector. The device 200 may comprise a sacrificial joint between the catheter 2 and the stent 1 that may dissolve in the presence of urine when an electrical charge is applied, similar to the mechanisms described in U.S. Pat. Nos. 5,122,136 and 5,643,254. The device 200 may use current resistance to soften or melt a connector, and since the connector may be internal to the catheter, no tissue may be affected by the temperature and the volume of body fluids flowing through the catheter may keep fluid temperatures within acceptable ranges. The device 200 may comprise shape memory component(s) and heating these components by electrical current can cause them change shape to release the catheter 2 and stent 1 from one another.

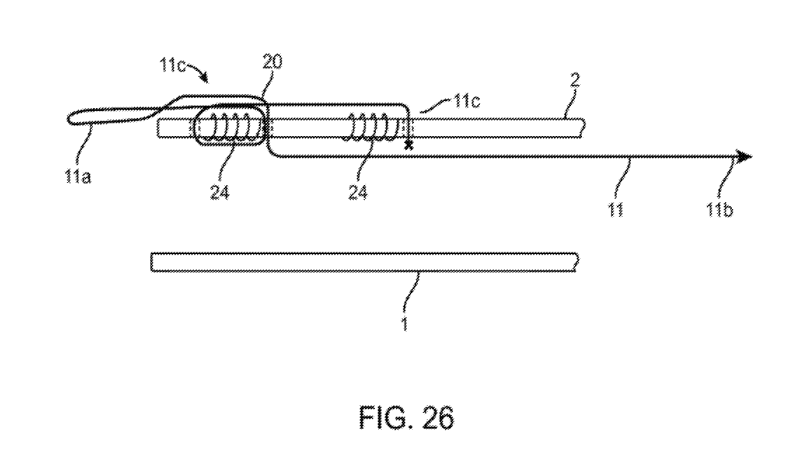

As shown in FIG. 26, the lock suture 11 may be tied down to the catheter 2 at multiple distal lock suture tie down locations 11c. The catheter 2 may be reinforced at the tie down locations 11c with reinforcements 24. The reinforcements 24 may comprise coil reinforced areas. These coil reinforced areas may be provided so that the lock suture 11 does not tear through the material of the catheter 2 under high load scenarios, as discussed further below. In some embodiments, a coil or other mode of reinforcement would also be located in a region of one or more of the lock suture proximal exit hole 20 or the lock suture distal entry hole 21 of the stent 1. For example, the aperture(s) surrounding lock suture proximal exit hole 20 and/or the lock suture distal entry hole 21 may be surrounded by a reinforcement ring or other reinforcement material. While catheter reinforcement is shown in FIG. 26, any of the convertible catheters and/or stents disclosed herein may have areas which are reinforced, particularly around the holes or apertures through which the lock or other sutures are passed through. Tensioning the lock or other suture may force the lock or other suture against the periphery of its respective hole or aperture and may damage the material of the convertible catheter and/or stent there; and, reinforcement may reduce the risk of such damage.

Lock Suture Distal Termination Methods: At least one or both ends of the lock suture 11 may be terminated toward the distal end of the catheter 2 to prevent separation of the stent-catheter junction under loading scenarios during delivery of the device.

The suture 11 may be terminated on pull wire 19 by passing through the braid of the suture 11 itself or tie knot to pull wire shaft. The knot or braid may slide longitudinally over the wire 18 as it is displaced or removed during a detachment event.

The knotted suture 11 may terminate within the lumen of the catheter 2 which may leverage against a small diameter hole. The hole which suture knot leverages against may be covered with an adhesive, marker band, and/or other polymeric sheathing.

A hypotube or marker band may be applied or crimped to the outer diameter, inner diameter, or embedded within the surface of the catheter 2 and/or stent 1 polymer. The metallic surfaces of the applied hypotube or marker band may be utilized for attaching suture material.

Lock Suture Hole Reinforcement: The holes punched (e.g., punched using a coring tool) through the wall of the catheter 2 and stent 1 in which the lock suture 11 passes through may require reinforcement to enhance the tear resistance of the thermoplastic used in many device applications which may cause the catheter 2 and/or stent 1 to soften at body temperature for optimal patient comfort. Locking suture materials usable in some applications may have the propensity to tear through the holes in the wall of the device under high load scenarios. A stiff metallic, polymeric, or fibrous braid or coil may be embedded, extruded, or laminated within the wall of one or more of the stent 1 or the catheter 2 to prevent such tearing. A segment of hypotube or other high strength material may be embedded, overlaid, or affixed near the holes of interest, but typically only near that region so as to not greatly impact the overall comfort characteristics of the device, so the suture may leverage against this stiff substrate under load.

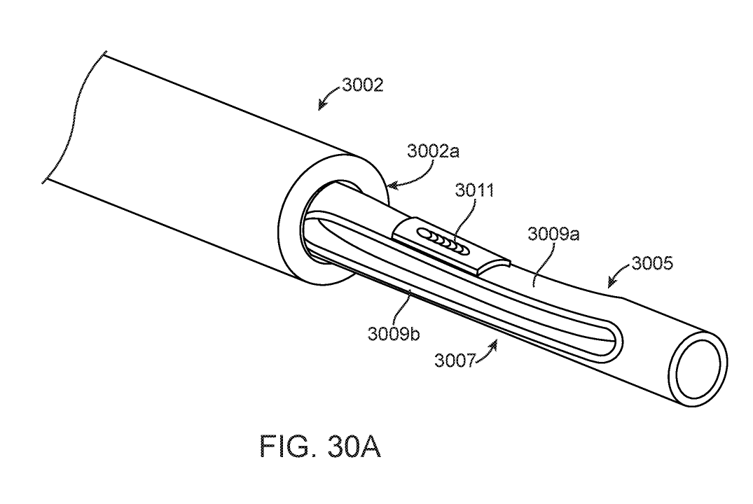

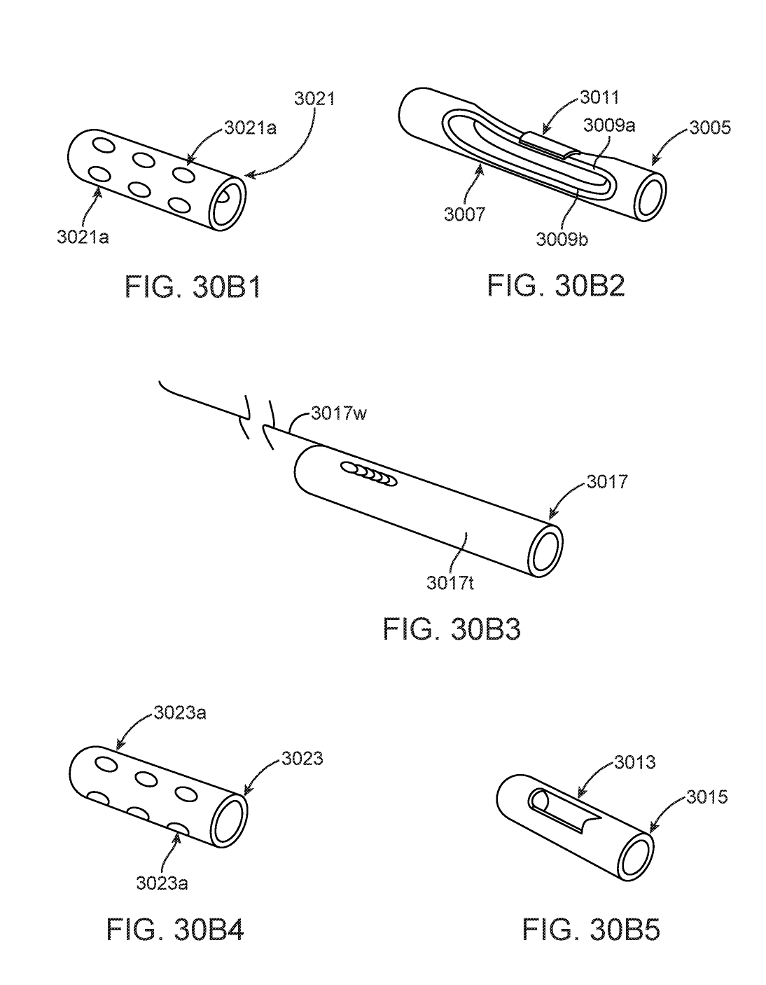

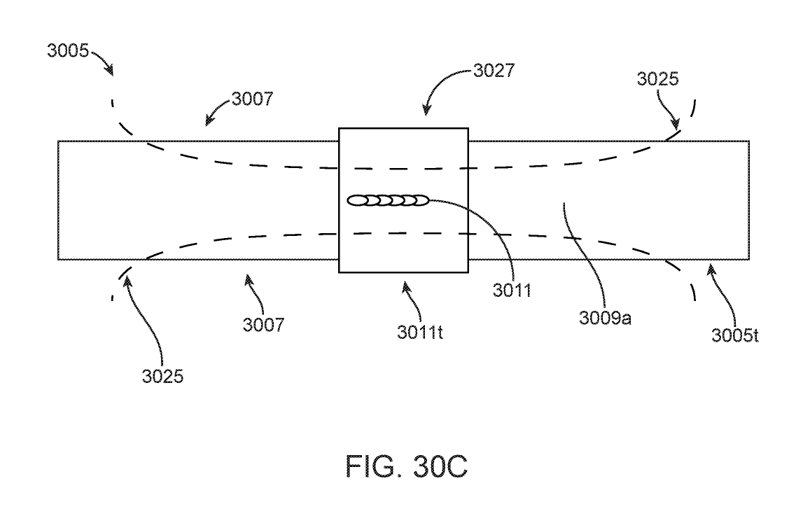

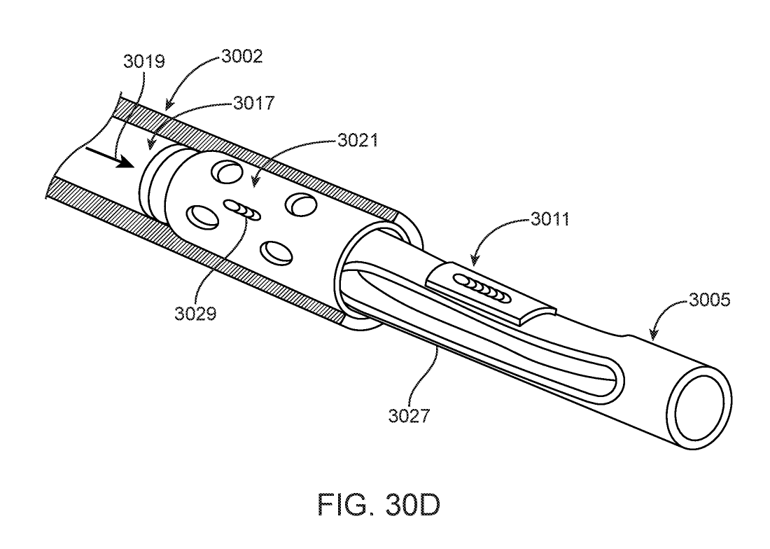

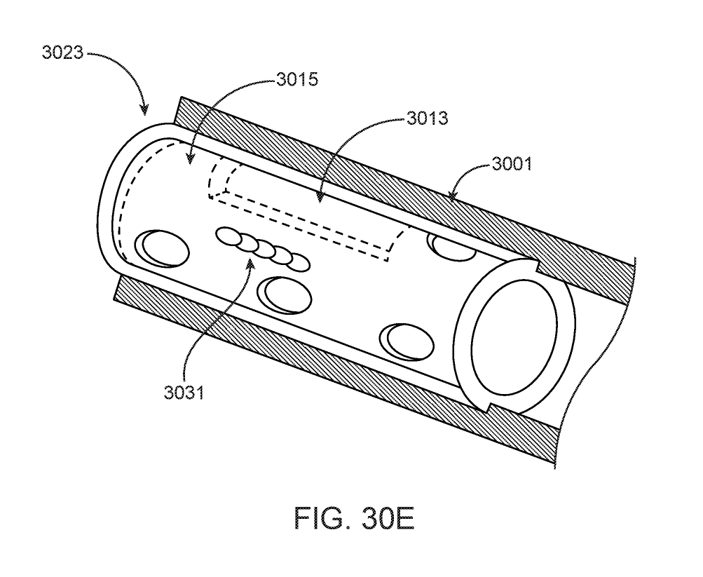

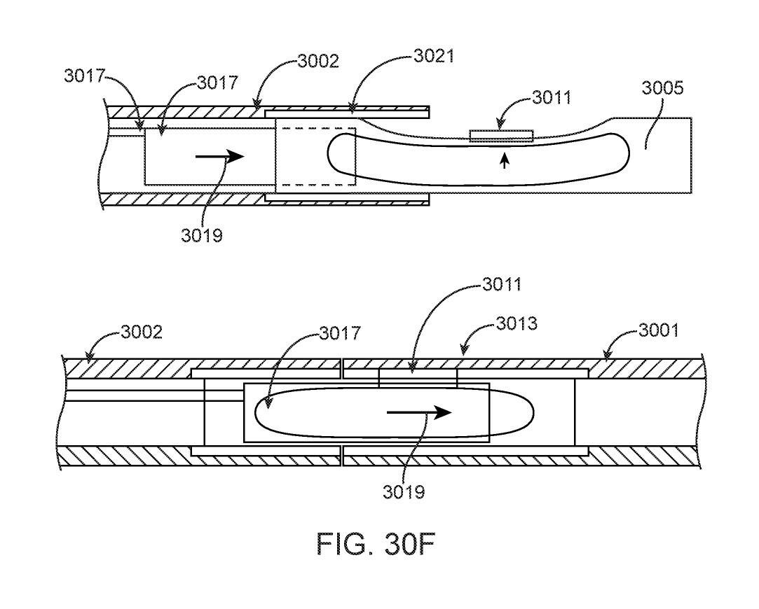

Referring to FIGS. 30A-30F, a further coupling and release mechanism is described. The coupling and release mechanism may comprise a proximal (inner) tube 3005 attached to the distal end of the catheter 3002 that has cut side windows 3007, such that the windows 3007 create two struts 3009a, 3009b, one strut having a rectangular steel key 3011 welded in the middle of the strut 3009a. The struts 3009a, 3009b may be cold formed or heat formed to be “bowed” downward toward the center axis of the tube 3005. The key 3011 may engage a corresponding rectangular window 3013 in an inner tube 3015 in the stent 3001 (the inner tube 3015 typically being a steel tube) and may provide both retention and torque-ability until released. An inner slide tube 3017 when moved distally (as shown by arrow 3019 in FIGS. 30D, 30F) may force the bowed strut 3009a upward, thus forcing the rectangular key 3011 into the lock window 3013. With the slide tube 3017 in place, the struts 3009a, 3009b of the inner lock tube 3005 may be contained in a “cast” between the slide tube 3017 and proximal inner tube 3005 embedded in the catheter 3002 and stent 3001 such that they should transmit rotational torque via the lock key 3011. Once the slide tube 3017 is retracted via a pull wire, the strut 3009a can return to its normally bowed condition, thus the lock key 3011 may fall out of the lock window 3013 and the stent 3001 may separate from the catheter 3002.

FIG. 30A shows the catheter side of the coupling and release mechanism, particularly the connector “key” or the proximal inner tube 3005 protruding from the inner lumen 3002a of the catheter 3002.

FIG. 30B1 shows the proximal outer tube 3021 for placement at the distal end of the inner lumen 3002a of the catheter 3002. The proximal outer tube 3021 may couple the proximal end of the proximal inner tube 3005 to the distal end of the catheter 3002. The proximal outer tube 3021 may have multiple holes 3021a (e.g., laser) drilled or cut for the material of the catheter 3002 (e.g., pellethane) to reflow into. The proximal outer tube may comprise a stainless tube, for example.

FIG. 30B2 shows the proximal inner (lock) tube 3005 comprising the lock key 3011 which may be (e.g., laser) welded, two (e.g., laser cut) windows 3007, where the windows 3007 may form opposing flexible struts 3009a, 3009b. The lock key 3011 may be welded at or near the center of the strut 3009a. The proximal end of the proximal inner lock tube 3005 may be (e.g., laser) welded to the inner lumen of the proximal outer tube 3021.

FIG. 30B3 shows the sliding tube assembly 3017. The sliding tube assembly 3017 may comprise a tube 3017t (typically nitinol, steel, or other metal) with a pull wire 3017w (typically nitinol, steel, or other metal) laser welded to the inside of the tube 3017.

FIG. 30B4 shows the distal outer tube 3023 which may be similar to the proximal outer tube 3021. The distal outer tube 3023 may have multiple holes 3023a (e.g., laser) drilled for the material of the stent 3001 (e.g., pellethane) to reflow into.

FIG. 30B5 shows the distal inner tube 3015 with the lock window 3013. The lock window 3013 may comprise a rectangular window (e.g., laser) cut in dimensions that will correspond to a loose fit around the lock key 3011. This tube 3015 may be (e.g., laser) welded to the inner lumen of the distal outer tube 3023, such that the lock window 3013 is covered and protected from reflow from the material of the stent 3001 (e.g., pellethane).

FIG. 30C shows the method of manufacture of the proximal inner lock tube assembly 3005. A short “lock key” tube 3011t may be slid over and (e.g., laser) welded (e.g., with one linear, axial weld) to a piece of tube 3005t. Two long windows 3007 are cut on each side of the tube 3005t with cutting paths 3025 avoiding the laser weld 3027 between the “lock key” tube 3011t and the piece of tube 3005, such that once completed, the lock key 3011 remains on one of the struts 3009a created by the two windows 3007. Subsequently, this strut 3009a may be bent down either beyond yield or heat treated to keep the lock key 3011 normally below the level of outer diameter of the tube 3005t to a significant degree.

FIG. 30D shows the proximal catheter connector assembly inside the distal end of the catheter 3002. The slide tube 3017 is shown in a retracted position on the inside of the (e.g., pellethane) catheter 3002. The proximal outer tube 3021 and proximal inner lock tube 3005 may be (e.g., laser) welded as shown with a weld 3029 and then formed into the distal end of the catheter 3002 by reflowing the material of the catheter 3002 (e.g., pellethane). The proximal inner lock tube 3005 may protrude distally such that the lock key 3011 is exposed by the correct distance corresponding the position of the lock window 3013 on the stent 3001. This distance may be, for example, 0.06″ or less from the distal end of the catheter 3002 to the proximal end of the lock key 3011.

FIG. 30E shows the distal connector assembly inside the proximal end of the stent 3001. The distal inner tube 3015 with the 3013 lock window may be (e.g., laser) welded to the inner lumen of the distal outer tube 3023 as shown by weld 3031, which may then be bonded to the inner lumen of the stent 3001 by reflow of the stent material (e.g., pellethane).

FIG. 30F shows the proximal connector cross section prior to being inserted into the stent 3001 (top) as well as the cross section of the catheter 3002 and stent 3001 after being connected (bottom). Since the lock window 3013 may not always be easily seen, a marker could be used to locate the proximal lock key 3011 properly in line with the lock window 3013, and once inserted, a tool may be used to advance the slide tube 3017. The slide tube 3017 may then force the strut 3009a of the proximal inner lock tube 3005 up, such that the lock key 3011 would be positioned in the lock window 3013.

Once the lock key 3011 is in the lock window 3013 with the slide tube 3005 below, the complete assembly may be locked together. The pull wire 3017w may be retracted to pull the slide tube 3005 back to release the stent 3002. While assembled, the struts 3009a, 3009b may be contain by both the inner and outer diameters of the slide tube 3017 and outer tubes 3021, 3023 such that the assembly is strong and can have near one-to-one torque-ability.

While the convertible catheter devices are described above as being used to deliver a nephrouretheral stent, the convertible catheter devices and their methods of use may be applicable for other anatomical structures as well. The dimensions and/or material properties of the convertible catheter devices may be modified to be appropriate for the other anatomical structures. For example, convertible catheter devices according to many embodiments may be suitable for use as a biliary stent to maintain the patency of a bile duct; and, the convertible catheter device usable to deliver a biliary stent may have a smaller proximal loop or a J-hook configuration of the proximal hook suitable for the shape of the gallbladder and/or gallbladder neck. In another example, convertible catheter devices according to many embodiments may be suitable for use as an ileal conduit catheter. While the convertible catheter devices adapted for use as a nephrouretheral stent may have a proximal to distal loop distance ranging from about 20 cm to about 28 cm, the convertible catheter devices adapted for use as ileal conduit catheters would have a longer loop to loop distance.

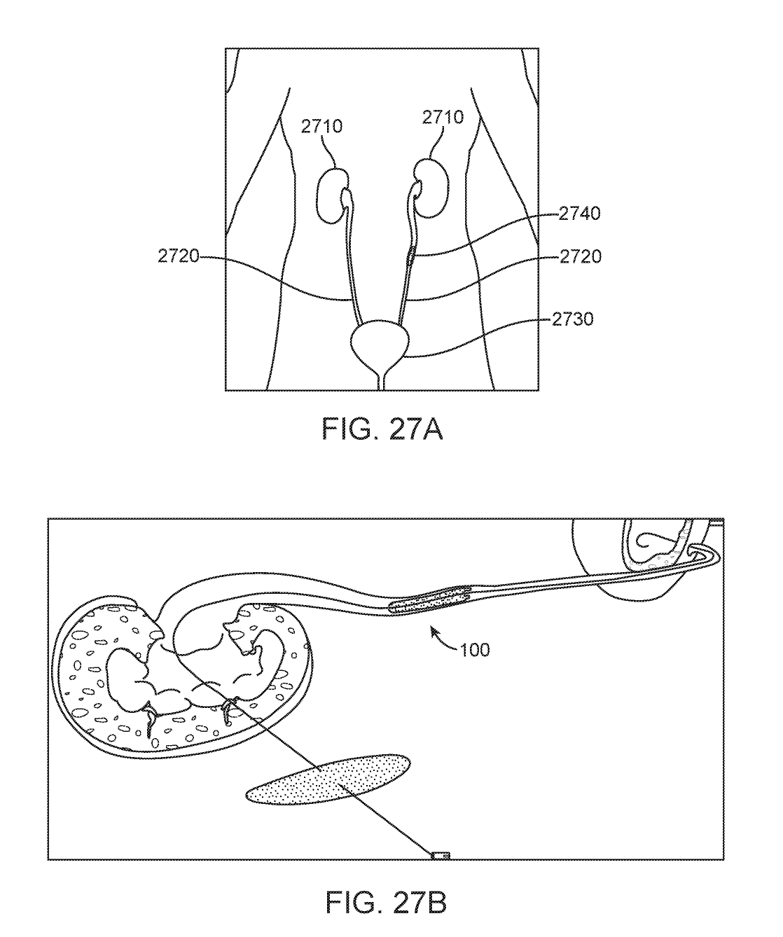

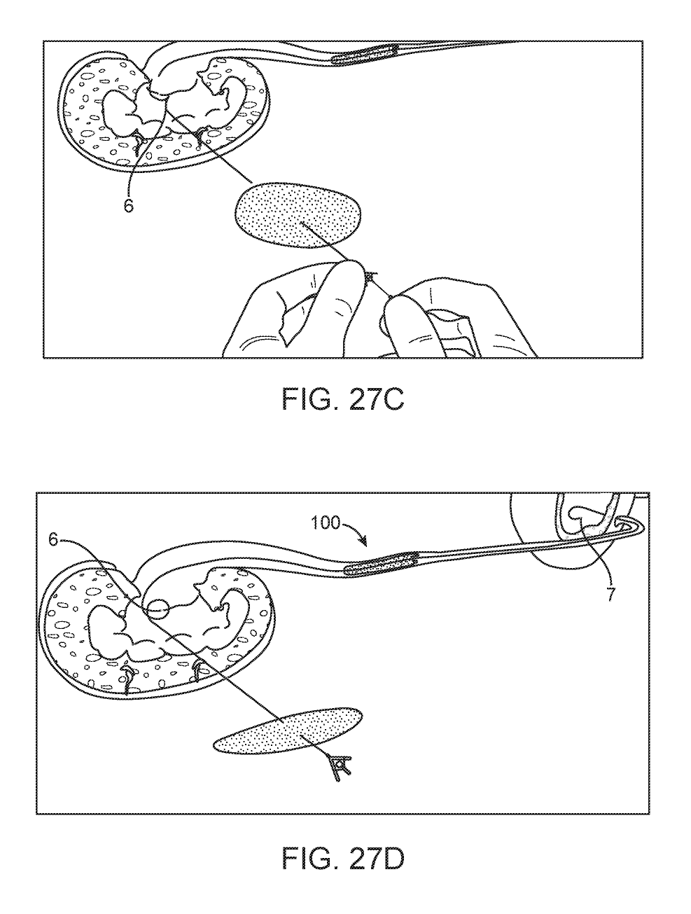

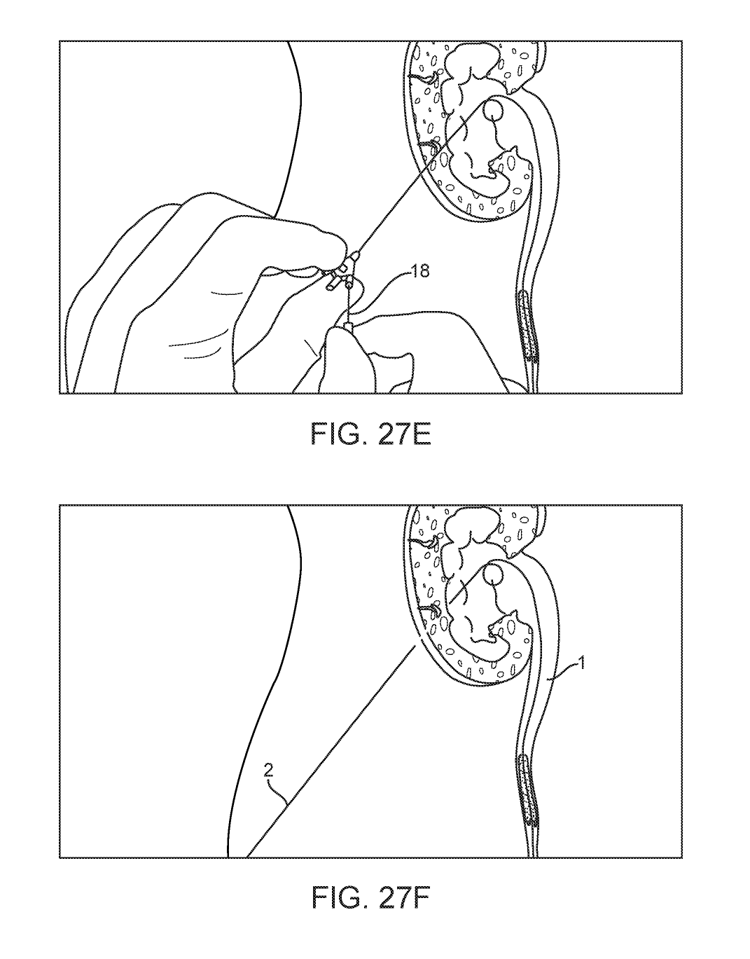

FIGS. 27A-27F illustrate an exemplary use of the system 200 to deliver and implant a nephrouretheral stent. FIG. 27A illustrates the target organs in such an operation, the target organs including the kidneys 2710, ureters 2720, and bladder 2730. FIG. 27A also illustrates an obstruction 2740 in one of the ureters 2720. For example, the obstruction may comprise a uretheral stone, a uretheral cyst, a benign or malignant grown the tissue adjacent the ureter, to name a few. In some embodiments, the stent member 1 can be biased so that it forms a proximal loop 6 and a distal loop 7, as illustrated in FIG. 17A-17B, for example. To insert the device 100 into the patient, the proximal loop 6 and distal loop 7 can be straightened out by inserting a hypotube or other straightener through the central lumen of the device, as described herein. FIG. 27B illustrates the insertion of the device 100 percutaneously into the patient (through skin S) with a hypotube or straightener in place, such that the stent member 1 is inserted with the loops 6, 7 straightened to facilitate advancement. FIGS. 27 C and 27D illustrate the stent member 1 assuming its biased, natural configuration with proximal and distal loops 6, 7 once the device 100 is inserted into the patient. Typically, the return to the looped configuration is achieved by the operator removing the hypotube or other straightener from the central lumen of the device. Additionally, referring to FIG. 17B, a loop suture 5c may need to be pulled to form the proximal loop 6. In some embodiments, the catheter portion 2 of the device 100 may be coupled to an external container to drain urine from the renal pelvis. Afterward, the patient may be released for at least 3 days as described above. Thereafter, the patient may return for a quick office visit so that the operator can decouple the stent member 1 and the catheter 2, according to the various release mechanisms described herein. For example, as illustrated in FIG. 27E, a lock wire 18 can be retracted, according to the details described herein. In some embodiments, the process of decoupling the stent member 1 and the catheter 2 takes less than 30 seconds. Accordingly, as illustrated in FIG. 27F, the catheter 2 is removed from the patient's body while the stent member 1 remains in the patient's body, with the proximal and distal loops 6, 7 being held in place in the renal pelvis and the bladder, respectively.

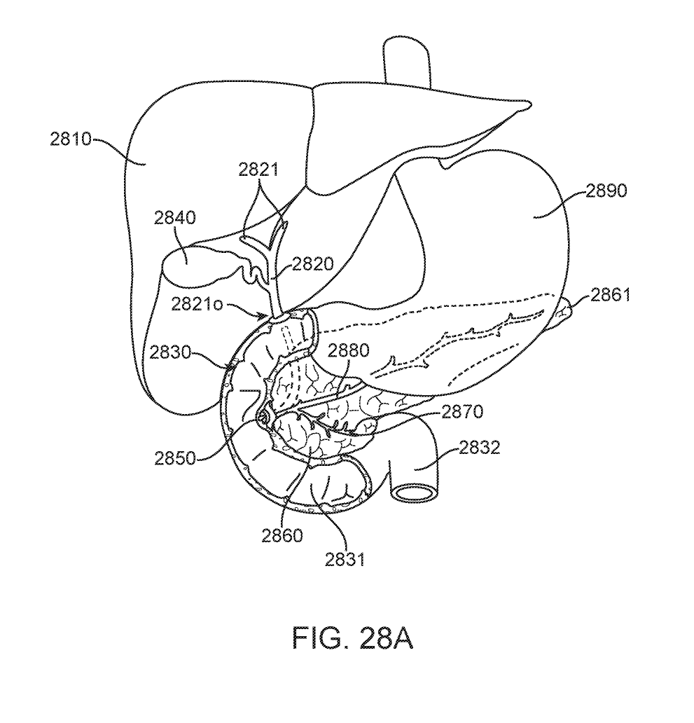

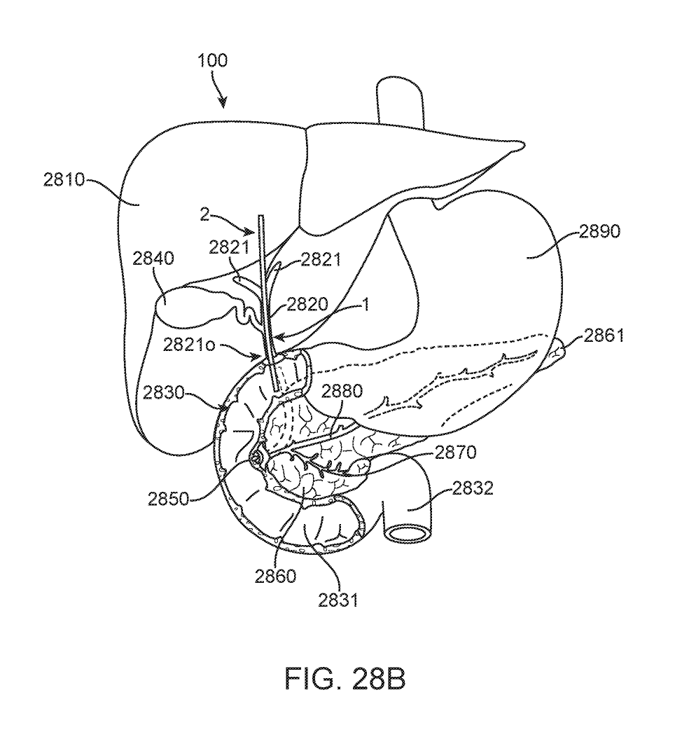

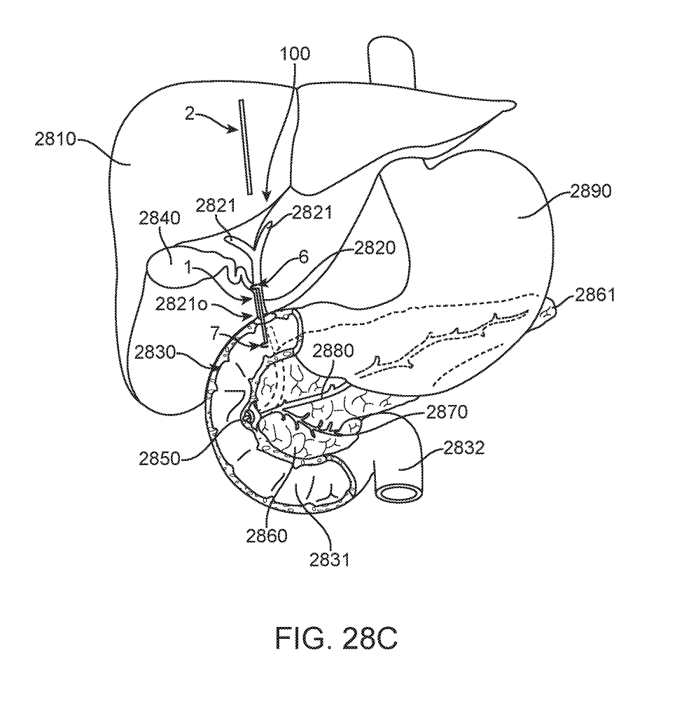

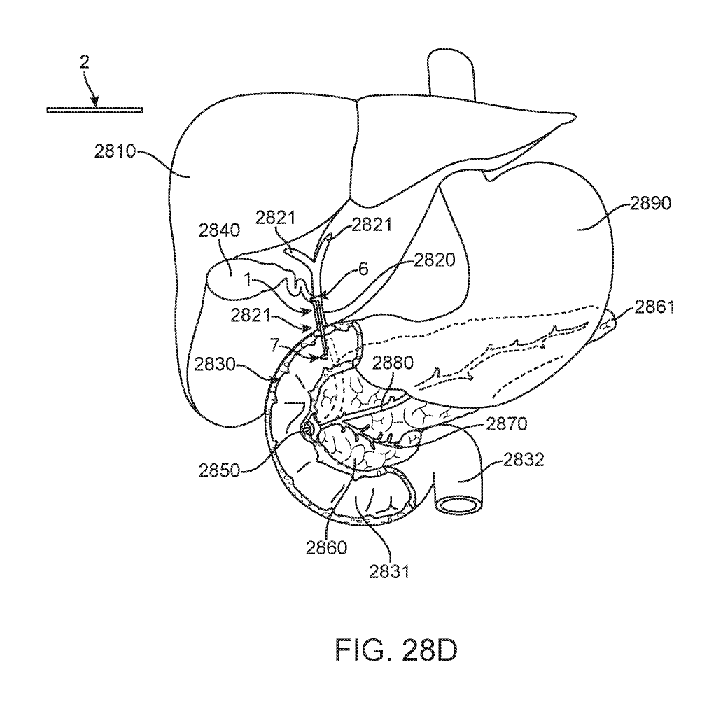

FIGS. 28A-28D illustrate an exemplary use of the system 200 to implant a convertible catheter/stent for use as a biliary stent to treat an obstruction 28210 in the common bile duct. FIG. 28A illustrates the relevant organs in such an operation, the organs including the liver 2810, common bile duct 2820, and the intestine 2830. Other surrounding anatomical structures shown include the gallbladder 2840, ampulla of vater 2850, pancreas 2860, duodenum 2831, small intestine 2832, accessory duct 2870, pancreatic duct 2880, tail of pancreas 2861, hepatic ducts 2821, and stomach 2890. The procedure to use the system 200 as a biliary stent is similar to the procedure described above for use of the system 200 as a nephrouretheral stent. FIG. 28A shows the relevant anatomy prior to the procedure. FIG. 28B illustrates insertion of the device 100 into the patient with a hypotube or straightener in place. Afterward, the operator may remove the hypotube or straightener, thereby allowing the proximal and distal loops 6, 7 to resume their looped configuration. FIG. 28C illustrates the stent member 1 assuming its biased, natural configuration with proximal and distal loops, 6, 7 once the device is inserted into the patient. The proximal loop 6 may be positioned in the common bile duct 2820 while the distal loop 7 may be positioned in the small intestine 2830, allowing bile to drain therethrough. The operator may then decouple the stent member 1 and catheter member 2 according to the various release mechanisms described herein. FIG. 28D illustrates the stent member 1 and the catheter member 2 decoupled, with the catheter member 2 removed from the patient's body and the stent member 1 remaining in the patient's body, with the proximal and distal loops 6, 7 being held in place in the common bile duct 2820 and intestine 2830, respectively. This procedure may be used generally where patency of the common bile duct 2820 is desired and maintained.

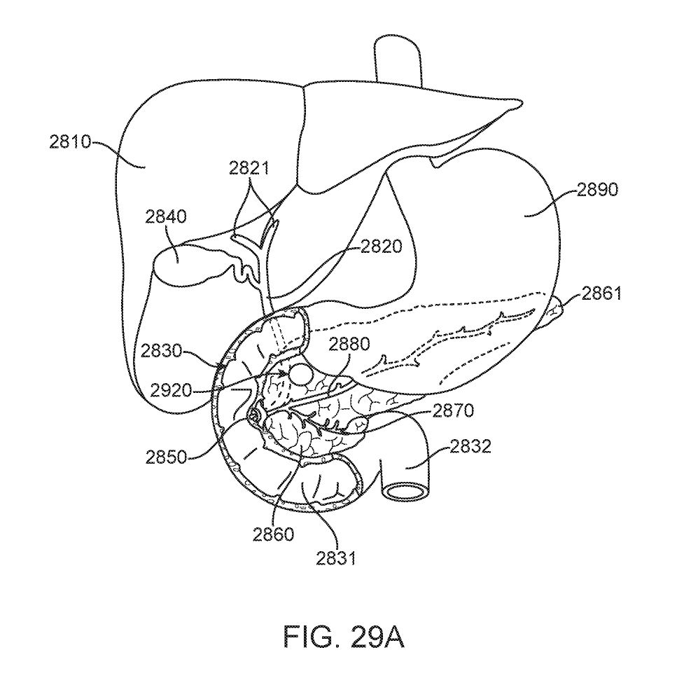

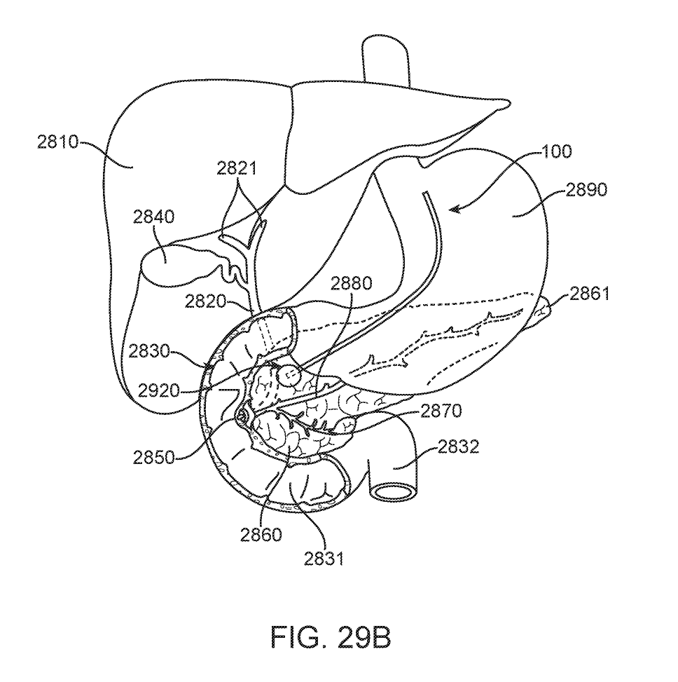

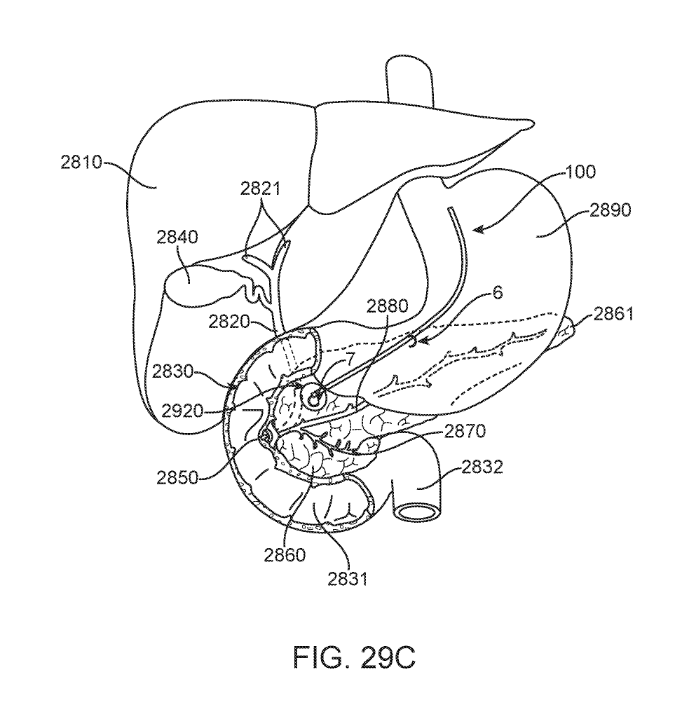

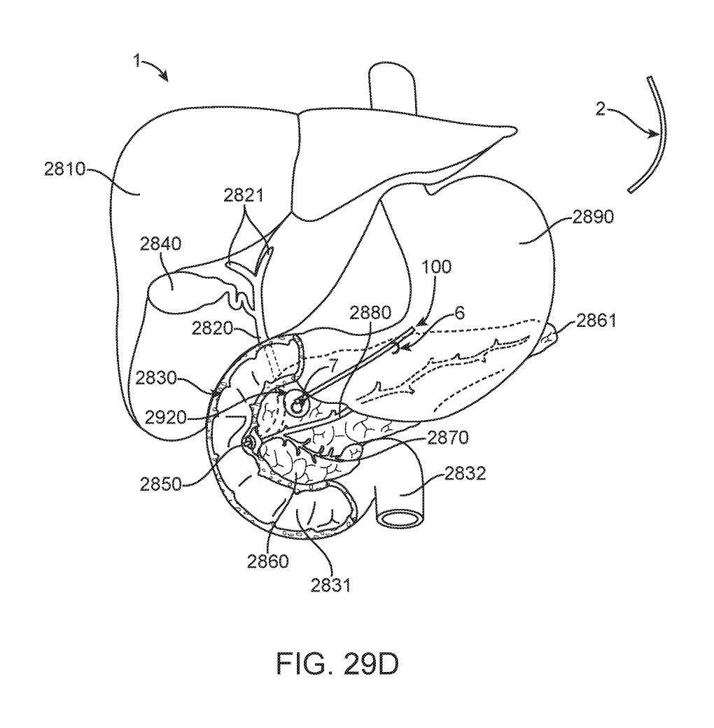

FIGS. 29A-29D illustrate an exemplary use of the system 200 to implant a convertible catheter/stent for use as a pancreatic stent. FIG. 29A illustrates the relevant anatomical structures in such an operation, including the stomach 2890, the pancreas 2860, and a pancreatic pseudocyst 2920 present in the pancreas 2860. A pancreatic pseudocyst 2920 is a cystic lesion which may cause abdominal pain, nausea, vomiting, a bloated feeling, trouble eating, and trouble digesting food. They are often caused by acute or chronic pancreatic, and may be treated by draining the pseudocyst 2920, such as into the stomach 2890. The procedure to use the system 200 as a pancreatic stent is similar to the procedures for use of the system as a nephrouretheral stent or as a biliary stent. FIG. 29A shows the relevant anatomy prior to the procedure. FIG. 29B illustrates insertion of the device 100 into the patient with a hypotube or straightener in place. Afterward, the operator may remove the hypotube or straightener. FIG. 29C illustrates the stent member 1 assuming its biased, natural configuration with proximal and distal loops 6, 7 once the device is inserted in the patient. The proximal loop 6 may be positioned in the stomach 2890 while the distal loop 7 may be positioned in the pseudocyst 2920, thereby allowing the pseudocyst 2920 to drain into the stomach 2890. Afterwards, the operator may decouple the stent member 1 and catheter member 2 according to the various release mechanisms described herein. FIG. 29D illustrates the stent member 1 and the catheter member 2 decoupled, with the catheter member 2 removed from the patient's body and the stent member 1 remaining in the patient's body, with the proximal and distal loops 6, 7 being held in place in the stomach 2890 and pseudocyst 2920, respectively. This procedure may be used to drain fluid from the pseudocyst into the stomach 2890 or intestine 2830.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of delivering a convertible stent, the method comprising:
advancing a stent delivery system through a percutaneous access site so that a distal end of a stent member of the stent delivery system is positioned in an intestine and a proximal end of the stent member is positioned in a common bile duct, wherein the distal end of the stent member forms a distal loop in the intestine;
actuating the proximal end of stent member to form the proximal end into a proximal loop in the common bile duct;
decoupling the stent member from a catheter body of the stent delivery system; and
retracting the catheter body from the percutaneous access site, leaving the stent member in place with the proximal end within the common bile duct and the distal end within the intestine.

2. The method of claim 1, wherein actuating the proximal end of the stent member to form the proximal loop comprises pulling a loop suture.

3. The method of claim 1, further comprising straightening one or more of the proximal or distal end of the stent member prior to advancing the stent delivery system.

4. The method of claim 3, wherein straightening the one or more of the proximal or distal end of the stent member comprises advancing a hypotube or other straightener through inner lumens of the stent member and the catheter body.

5. The method of claim 4, further comprising removing the hypotube or other straightener, thereby allowing the one or more of the proximal or distal end of the stent member to form the proximal loop or the distal loop, respectively.

6. The method of claim 1, further comprising leaving the stent member and the catheter body of the stent delivery system in place until hemostasis is achieved, before decoupling the stent member from the catheter body.

7. The method of claim 1, wherein at least a portion of the catheter body is coated with a hemostatic agent.

8.-36. (canceled)
